(19) United States
(12) Patent Application Publication
(10) Pub. No.: US 2025/0095773 A1
(43) Pub. Date: Mar. 20, 2025

(54) METHODS AND SYSTEMS FOR DEVELOPING CHROMATOGRAPHY PROTOCOLS

(71) Applicant: Regeneron Pharmaceuticals, Inc., Tarrytown, NY (US)

(72) Inventors: Nimish GUPTA, West Harrison, NY (US); Christopher COWAN, Briarcliff Manor, NY (US); John MATTILA, Tarrytown, NY (US)

(73) Assignee: Regeneron Pharmaceuticals, Inc., Tarrytown, NY (US)

(21) Appl. No.: 18/887,534

(22) Filed: Sep. 17, 2024

Related U.S. Application Data
(60) Provisional application No. 63/583,474, filed on Sep. 18, 2023.

(51) Int. Cl. G16B 15/30; B01D 15/38

(52) U.S. Cl. CPC G16B 15/30; B01D 15/3804

(57) ABSTRACT

A method of purifying a target molecule may include introducing a load including a high molecular weight species concentration (% HMW) to a chromatography apparatus comprising sartobind phenyl chromatography media. A method of generating a chromatography protocol, may include identifying chromatography loading parameters, identifying chromatography performance criteria. The method of generating the chromatography protocol may include selecting combinations of test values of the loading parameters, and conducting a chromatography run for each combination of the set of test values combinations, thereby generating actual performance criteria values corresponding to each combination of the set of test value combinations.

### CROSS-REFERENCE TO RELATED APPLICATIONS

This application claims priority to U.S. Provisional Patent Application No. 63/583,474, filed on Sep. 18, 2023, which is hereby incorporated by reference in its entirety.

### FIELD OF DISCLOSURE

The present disclosure relates to systems and methods for developing and implementing chromatography protocols for biopharmaceutical product production. Some aspects of the present disclosure relate to systems and methods for developing hydrophobic interaction (HIC) chromatography protocols.

### INTRODUCTION

Biopharmaceutical products (e.g., antibodies, antibody-drug conjugates, fusion proteins, adeno-associated viruses (AAVs), proteins, tissues, cells, polypeptides, or other therapeutic products of biological origin) are increasingly being used in the treatment and prevention of infectious diseases, genetic diseases, autoimmune diseases, and other ailments. Production of the biopharmaceutical products requires chromatography to purify, characterize, and validate the products.

The production of biopharmaceutical products may include, for example, affinity chromatography (e.g., Protein A or Protein L), ion-exchange chromatography, size-exclusion chromatography, reverse phase chromatography, fast protein liquid chromatography, high-performance liquid chromatography, countercurrent chromatography, periodic counter-current chromatography, chiral chromatography, and mixed-mode chromatography, and hydrophobic interaction chromatography.

Conventional methods of developing chromatography protocols, and optimizing parameters of the chromatography protocols, are time and labor intensive, and waste a large amount of reagents and product. The time and labor required also results in a large cost associated with modifying an existing chromatography protocol. The use of non-optimized chromatography protocols themselves can also lead to waste of reagents and/or products, which increases the costs associated with the production of biopharmaceutical products.

### SUMMARY

Aspects of the present disclosure may be directed to a method of generating a chromatography protocol for a target molecule. The target molecule may be an antibody-drug conjugate. The method may include identifying chromatography loading parameters, identifying chromatography performance criteria, generating a domain of potential predictive models relating the chromatography loading parameters to the chromatography performance criteria, selecting combinations of test values of the loading parameters, wherein the selected combinations of test values form a set of test value combinations, conducting a chromatography run for each combination of the set of test values combinations, thereby generating actual performance criteria values corresponding to each combination of the set of test value combinations, and ranking each predictive model, of the domain of predictive models, based on a correlation of performance criteria values predicted by the model to the actual performance criteria values.

In some aspects of the present disclosure, the chromatography loading parameters may include a loading buffer salt concentration, a loading density, a high molecular weight species content of the load, or a combination thereof. The chromatography performance criteria may include a high molecular weight species content reduction, yield of a target molecule, or a combination thereof.

In some aspects of the present disclosure, the method may further include selecting a predictive model that has the highest rank, and using the selected model to determine values of the chromatography loading parameters for the chromatography protocol. The selected model may predict that the values of the chromatography loading parameters for the chromatography protocol correspond to one or more target performance criteria values.

In some aspects of the present disclosure, the method may further include developing a desirability metric that includes a quantitative relationship between performance criteria. The desirability metric may be calculated as a composite of two or more performance criteria, wherein each performance criterion is assigned a weight that contributes to the desirability metric. The two or more performance criteria may include a high molecular weight species content reduction and a yield of a target molecule, wherein each performance criterion is equally weighted.

Aspects of the present disclosure may also be directed to a method of generating a chromatography protocol for a target molecule. The target molecule may be an antibody-drug conjugate. The method may include identifying a first chromatography parameter and a second chromatography parameter, identifying a first performance criterion and a second performance criterion, selecting first test values for the first chromatography parameter, selecting second test values for the second chromatography parameter, identifying a chromatography media, generating first performance criterion values, wherein each first performance criterion value corresponds to a combination of a first test value and a second test value, generating second performance criterion values, wherein each second performance criterion value corresponds to a combination of a first test value and a second test value, generating a first pool of multivariate models, wherein each multivariate model of the first pool of multivariate models relates the first and second chromatography parameters to the first performance criterion, generating a second pool of multivariate models, wherein each multivariate model of the second pool of multivariate models relates the first and second chromatography parameters to the second performance criterion, generating first projected performance criterion values using each multivariate model of the first pool of multivariate models, wherein each first projected performance criterion value corresponds to a combination of a first test value and a second test value, generating second projected performance criterion values using each multivariate model of the second pool of multivariate models, wherein each second projected performance criterion value corresponds to a combination of a first test value and a second test value, determining a coefficient of determination for a multivariate model in the first pool of multivariate models, and determining a coefficient of determination for a multivariate model in the second pool of multivariate models.

In some aspects of the present disclosure, identifying the chromatography media may include performing a first chromatography run on a first chromatography media to generate a first desirability value corresponding to the first chromatography media, performing a second chromatography run on a second chromatography media to generate a second desirability value corresponding to the second chromatography media, and selecting the chromatography media with the that corresponds to the greatest desirability value. The first chromatography run and the second chromatography run may be conducted with the same media density and loading buffer composition. The first desirability value may be calculated based on one or more performance criteria values of the first chromatography run, and the second desirability value may be calculated based on one or more performance criteria values of the second chromatography run. The first chromatography parameter may be a column loading of a HIC media (g/L), the second chromatography parameter may be a citrate salt concentration of a loading buffer, the first performance criterion may be a yield, and the second performance criterion may be a quantification of the reduction of an impurity. Generating the first pool of multivariate models may include identifying a domain of potential multivariate models, calculating a variance inflation factor for each potential multivariate model of the domain of potential multivariate models, and selecting all potential multivariate models with a variance inflation factor less than or equal to a collinearity threshold to generate the first pool of multivariate models.

Aspects of the present disclosure may also be directed to a method of purifying a target molecule. The target molecule may be an antibody-drug conjugate. The method may include introducing a load including a high molecular weight species concentration (% HMW) of approximately 3 percent to approximately 20 percent to a chromatography apparatus comprising sartobind phenyl chromatography media, wherein the load is introduced at a density of approximately 10 grams of load per liter of total volume of the chromatography apparatus to approximately 40 grams per liter, and wherein the load comprises the target molecule and approximately 5 mM to approximately 200 mM of citrate, and passing an eluate comprising the target molecule from the chromatography apparatus, wherein a yield of the target molecule in the eluate is at least approximately 70%, and wherein a difference of the % HMW of load and a % HMW of the eluate is at least approximately 2%.

In some aspects of the present disclosure, the antibody-drug conjugate may include a drug conjugated to an antibody via lysine conjugation. A % HMW of the load may be approximately 7% to approximately 15%. The load may be introduced at a density of approximately 20 grams per liter to approximately 30 grams per liter. The load may comprise approximately 110 mM to approximately 175 mM citrate. The yield of the target protein in the eluate may be approximately 75% to approximately 95%. A difference of the % HMW of load and a % HMW of the eluate may be approximately 3% to approximately 8%. The concentration of the target protein in the eluate may be between approximately 70% to approximately 85% of the concentration of the target protein in the load. The antibody-drug conjugate may include a cleavable maytansinoid. The antibody-drug conjugate may include an IgG 4 antibody.

### BRIEF DESCRIPTION OF THE DRAWINGS

The accompanying drawings, which are incorporated in and constitute a part of this specification, illustrate various exemplary embodiments, and together with the description, serve to explain the principles of the disclosed embodiments. Any features of an embodiment or example described herein, e.g., composition, formulation, method, etc., may be combined with any other embodiment or example, and all such combinations are encompassed by the present disclosure. Moreover, the described systems and methods are neither limited to any single aspect nor embodiment thereof, nor to any combinations or permutations of such aspects and embodiments. For the sake of brevity, certain permutations and combinations are not discussed and/or illustrated separately herein.

FIG. 1 depicts, in flow-chart form, an exemplary method for developing a chromatography protocol, according to aspects of the present disclosure;

FIG. 2 depicts, in flow-chart form, an exemplary method for developing a chromatography protocol, according to aspects of the present disclosure;

FIG. 3 depicts a plot of performance criteria associated with different chromatography media;

FIG. 4 depicts a series of plots of performance criteria versus chromatography loading parameters, according to aspects of the present disclosure;

FIG. 5 depicts a chromatogram of an exemplary chromatography protocol, according to aspects of the present disclosure;

FIG. 6A depicts a mass spectrogram of a sample, prior to a chromatography process, according to aspects of the present disclosure;

FIG. 6B depicts a mass spectrogram of the sample of FIG. 6A, after a chromatography process, according to aspects of the present disclosure;

FIG. 7A depicts a plot of a particle size distribution of a sample, prior to a chromatography process, according to aspects of the present disclosure;

FIG. 7B depicts a plot of a particle size distribution of the sample of FIG. 7A, after a chromatography process, according to aspects of the present disclosure;

FIG. 8 depicts a bioassay plot, according to aspects of the present disclosure; and

FIG. 9 depicts a binding assay plot, according to aspects of the present disclosure.

### DETAILED DESCRIPTION

Unless defined otherwise, all technical and scientific terms used herein have the same meaning as is commonly understood by one of ordinary skill in the art to which this disclosure belongs. Although any suitable methods and materials, e.g., similar or equivalent to those described herein, can be used in the practice or testing of the present disclosure, particular example methods are now described. All publications mentioned are hereby incorporated by reference.

As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” For the terms “for example” and “such as,” and grammatical equivalences thereof, the phrase “and without limitation” is understood to follow unless explicitly stated otherwise.

As used herein, the term “approximately” is meant to account for variations due to experimental error. When applied to numeric values, the term “approximately” may indicate a variation of +/−5% from the disclosed numeric value, unless a different variation is specified. As used herein, the singular forms “a,” “an,” and “the” include plural referents unless the context clearly dictates otherwise. Further, all ranges are understood to be inclusive of endpoints, e.g., from 1 mm to 5 mm would include 1 mm and 5 mm and all distances or lengths between 1 mm and 5 mm.

It should be noted that all numeric values disclosed or claimed herein (including all disclosed values, limits, and ranges) may have a variation of +/−5% from the disclosed numeric value unless a different variation is specified.

As used herein, the term “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, antibody-like molecules, nanobodies, recombinant antibody chimeras, cytokines, chemokines, peptide hormones, and the like. The term “polypeptide” as used herein may refer to any amino acid polymer having more than approximately 20 amino acids covalently linked via amide bonds. Proteins contain one or more amino acid polymer chains (e.g., polypeptides). Thus, a polypeptide may be a protein, and a protein may contain multiple polypeptides to form a single functioning biomolecule.

A target molecule may include any polypeptide, protein, or other molecule that is desired to be isolated, purified, characterized, or otherwise prepared. Target molecules may include polypeptides produced by a cell, including antibodies. Target molecules may include antibodies that have undergone post-translation modifications, such as, for example lysine based conjugation. Lysine based conjugation may be used to covalently connect an antibody produced by a cell to one or more other pharmaceutically active compounds. Antibodies that have been covalently connected to one or more other pharmaceutically active compounds may be referred to as antibody-drug conjugates, and are discussed in further detail below.

Target molecules (e.g., peptides or antibodies) may be produced using recombinant cell-based production systems, such as the insect bacculovirus system, yeast systems (e.g., Pichia sp.), or mammalian systems (e.g., CHO cells and CHO derivatives like CHO-K1 cells). The term “cell” includes any cell that is suitable for expressing a recombinant nucleic acid sequence. Cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, bacculovirus-infected insect cells, Trichoplusiani, etc.), non-human animal cells, human cells, or cell fusions such as, for example, hybridomas or quadromas. In some embodiments a cell may be a human, monkey, ape, hamster, rat, or mouse cell. In some embodiments, a cell may be eukaryotic and may be selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, W138, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, a cell may comprise one or more viral genes, e.g. a retinal cell that expresses a viral gene (e.g., a PER.C6™ cell).

The term “antibody,” as used herein, includes immunoglobulins comprised of four polypeptide chains: two heavy (H) chains and two light (L) chains inter-connected by disulfide bonds. Typically, antibodies have a molecular weight of over 100 kDa, such as between 130 kDa and 200 kDa, such as approximately 140 kDa, 145 kDa, 150 kDa, 155 kDa, or 160 kDa. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3).

A class of immunoglobulins called Immunoglobulin G (IgG), for example, is common in human serum and comprises four polypeptide chains—two light chains and two heavy chains. Each light chain is linked to one heavy chain via a cystine disulfide bond, and the two heavy chains are bound to each other via two cystine disulfide bonds. Other classes of human immunoglobulins include IgA, IgM, IgD, and IgE. In the case of IgG, four subclasses exist: IgG 1, IgG 2, IgG 3, and IgG 4. Each subclass differs in their constant regions, and as a result, may have different effector functions. In some embodiments described herein, a target molecule may comprise a polypeptide including IgG, such as, for example, IgG 4.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

The term “drug”, as used herein, may refer to any compound possessing a desired biological activity. The desired biological activity may include activity useful in the diagnosis, cure, mitigation, treatment, or prevention of a disease in human or other animal. As used herein, a drug may include a reactive functional group available that allows for conjugation of the drug to an antibody. In some embodiments, an antibody is linked to a drug via a linker, and the drug has a functional group that can form a bond with the linker. For example, a drug may have a reactive group, e.g., an amino group, a carboxyl group, a thiol group, a hydroxyl group, or a ketone group, etc., that can form a bond with a linker. A drug may be any cytotoxic drug which inhibits cell growth or immunosuppression. For example, a drug may include anti-tubulin agents, DNA minor groove binding agents, DNA replication inhibitors, alkylating agents, antibiotics, folic acid antagonists, antimetabolites, chemotherapy sensitizers, topoisomerase inhibitors, vinca alkaloids, steroids, vitamins, agonists, antagonists, ligands, signaling molecules, checkpoint inhibitors, oligonucleotides including siRNAs, peptides, immune system stimulators/suppressors, hormones, anti-virals, anti-fungals, radionuclides, chelators, oligosaccharides, ligands, or other suitable compounds having a inhibitory effect on cell growth. Examples of particularly useful cytotoxic drugs include, DNA minor groove binding agents, DNA alkylating agents, tubulin inhibitors, auristatins, camptothecins, docamycin/duocarmycins, etoposides, maytansines, maytansinoids (e.g. DM1, DM4, M114), taxanes, benzodiazepines or benzodiazepine containing drugs (e.g. pyrrolo[1,4]benzodiazepines (PBDs), indolinobenzodiazepines and oxazolidinobenzodiazepines), and vinca alkaloids. In one or more embodiments, an antibody drug conjugate may include a cleavable maytansinoid.

An antibody drug conjugate may refer to any suitable substance comprising an antibody chemically linked to a drug (e.g., an antibody covalently bonded to a drug). An antibody may be conjugated to a drug via any suitable means, e.g., random lysine conjugation, to form an antibody-drug conjugate. Other suitable means for conjugating an antibody to a drug include, but are not limited to, cysteines (e.g., engineered cysteines), enzyme conjugation (e.g., sortase), transglutaminase, a formyl glycine generating enzyme, non-covalent conjugation, carbonates, unnatural amino acids, engineered azides, and inter-chain cysteine re-bridging linkers.

An antibody-drug conjugate may comprise a linker between a drug and an antibody. The linker may be a degradable or non-degradable linker. Degradable linkers may include, for example, enzyme-degradable linkers, including peptidyl-containing linkers that can be degraded by protease (e.g. lysosomal protease or endosomal protease) in a cell, or sugar linkers, for example, glucuronide-containing linkers that can be degraded by glucuronidase. Peptidyl linkers may include, for example, dipeptides, such as valine-citrulline, phenylalanine-lysine or valine-alanine. Other suitable degradable linkers include, for example, pH sensitive linkers (e.g. linkers that are hydrolyzed at a pH of below 5.5, such as hydrazone linkers), linkers that are degraded under reducing conditions (e.g. disulfide-bond linkers), glycosidase-cleavable linkers, phosphatase-cleavable linkers, and photo-responsive linkers. A non-degradable linker may typically release a drug under conditions in which the antibody is hydrolyzed by protease.

Antibody-drug conjugates of the present disclosure may include one or more types of antibody, such as, for example, a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody, or an antibody-drug conjugates. In one aspect, the antibody is an IgG1 antibody. In one aspect, the antibody is an IgG2 antibody. In one aspect, the antibody is an IgG4 antibody. In one aspect, the antibody is a chimeric IgG2/IgG4 antibody. In one aspect, the antibody is a chimeric IgG2/IgG1 antibody. In one aspect, the antibody is a chimeric IgG2/IgG1/IgG4 antibody. In one aspect, the antibody-drug conjugate includes an IgG 4 antibody.

In some aspects, antibody-drug conjugates of the present disclosure may include one or more types of antibody selected from a group consisting of an anti-Programmed Cell Death 1 antibody (e.g., an anti-PD1 antibody as described in U.S. Pat. Appln. Pub. No. US2015/0203579A1), an anti-Programmed Cell Death Ligand-1 (e.g. an anti-PD-L1 antibody as described in U.S. Pat. Appln. Pub. No. US2015/0203580A1), an anti-D114 antibody, an anti-Angiopoetin-2 antibody (e.g., an anti-ANG2 antibody as described in U.S. Pat. No. 9,402,898), an anti-Angiopoetin-Like 3 antibody (e.g. an anti-AngPt13 antibody as described in U.S. Pat. No. 9,018,356), an anti-platelet derived growth factor receptor antibody (e.g. an anti-PDGFR antibody as described in U.S. Pat. No. 9,265,827), an anti-Prolactin Receptor antibody (e.g., anti-PRLR antibody as described in U.S. Pat. No. 9,302,015), an anti-Complement 5 antibody (e.g., an anti-C5 antibody as described in U.S. Pat. Appln. Pub. No US2015/0313194A1), an anti-TNF antibody, an anti-epidermal growth factor receptor antibody (e.g., an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (e.g., an anti-PCSK9 antibody as described in U.S. Pat. No. 8,062,640 or U.S. Pat. Appln. Pub. No. US2014/0044730A1), an anti-Growth And Differentiation Factor-8 antibody (e.g., an anti-GDF8 antibody, also known as anti-myostatin antibody, as described in U.S. Pat No. 8,871,209 or 9,260,515), an anti-Glucagon Receptor (e.g., anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. Appln. Pub. No. US2014/0271681A1 or U.S. Pat No. 8,735,095 or 8,945,559), an anti-interleukin 6 receptor antibody (e.g., an anti-IL6R antibody as described in U.S. Pat. Nos. 7,582,298, 8,043,617 or 9,173,880), an anti-interleukin 33 (e.g., anti-IL33 antibody as described in U.S. Pat. Appln. Pub. Nos. US2014/0271658A1 or US2014/0271642A1), an anti-Respiratory syncytial virus antibody (e.g., anti-RSV antibody as described in U.S. Pat. Appln. Pub. No. US2014/0271653A1), an anti-Cluster of differentiation 3 (e.g., an anti-CD3 antibody, as described in U.S. Pat. Appln. Pub. Nos. US2014/0088295A1 and US20150266966A1, and in U.S. Application No. 62/222,605), an anti-Cluster of differentiation 20 (e.g., an anti-CD20 antibody as described in U.S. Pat. Appln. Pub. Nos. US2014/0088295A1 and US20150266966A1, and in U.S. Pat. No. 7,879,984), an anti-Cluster of Differentiation-48 (e.g., anti-CD48 antibody as described in U.S. Pat. No. 9,228,014), an anti-Fel d1 antibody (e.g., as described in U.S. Pat. No. 9,079,948), an anti-Middle East Respiratory Syndrome virus (e.g., an anti-MERS antibody), an anti-Ebola virus antibody (e.g., Regeneron's REGN-EB3), an anti-CD19 antibody, an anti-CD28 antibody, an anti-IL1 antibody, an anti-IL2 antibody, an anti-IL3 antibody, an anti-IL4 antibody, an anti-IL5 antibody, an anti-IL6 antibody, an anti-IL7 antibody, an anti-Erb3 antibody, an anti-Zika virus antibody, an anti-Lymphocyte Activation Gene 3 (e.g., anti-LAG3 antibody or anti-CD223 antibody) and an anti-Activin A antibody. Each U.S. patent and U.S. patent publication mentioned in this paragraph is incorporated by reference in its entirety.

In some aspects, antibody-drug conjugates of the present disclosure may include one or more types of antibody selected from the group consisting of an anti-CD3×anti-CD20 bispecific antibody, an anti-CD3×anti-Mucin 16 bispecific antibody, and an anti-CD3×anti-Prostate-specific membrane antigen bispecific antibody. In some aspects, the target molecule includes an antibody selected from the group consisting of alirocumab, sarilumab, fasinumab, nesvacumab, dupilumab, trevogrumab, evinacumab, and rinucumab.

The term “impurity”, as used herein refers to foreign or objectionable molecules, including nucleic acids, proteins, and other compounds, that are not the target molecule of a chromatography operation. Exemplary impurities include host cell proteins, variants of target molecules (e.g., aggregates, deamidated species, and/or misconjugated antibody-drug conjugates), low molecular weight species and fragments, proteins that are part of an absorbent used for affinity chromatography (e.g. Protein A or Protein L), excess free linker-payload (e.g., drug components of an antibody-drug conjugate that are not bound to an antibody), excess reagents from the conjugation process (e.g., organic solvents), endotoxins, and viruses.

As noted above, chromatography techniques, may be used to separate different components of a mixture, to purify target molecules (e.g., antibody-drug conjugates), and/or to characterize the content of a mixture. However, in some instances, high molecular weight species that are not included as biopharmaceutical products (e.g., host cell proteins, aggregates, other impurities), may co-elute with the biopharmaceutical products. Moreover, as drug products including the biopharmaceutical products and other high molecular weight species are subjected to stress (e.g., time and/or extreme temperatures), the percentage of high molecular weight species in the drug product may increase. Therefore, drug product formulations (e.g., formulations including a biopharmaceutical product that are administered to a subject) with a lower percentage of high molecular weight species may exhibit an improved shelf life, compared to other formulations.

Accordingly, while some chromatography protocols, e.g., HIC protocols, may result in a drug product with an acceptable high molecular weight species content, there exists a need for improved chromatography protocols that further reduce the amount of number of high molecular weight species that co-elute with biopharmaceutical products. Improved chromatography protocols may result in a drug product formulation with a decreased of impurities, compared to formulations developed by conventional chromatography methods. Chromatography protocols of the present disclosure may reduce the concentration of impurities (e.g., high molecular weight species) in a sample. In addition or alternatively, chromatography protocols of the present disclosure may increase the concentration of a target molecule.

Various parameters of a chromatography protocol may be adjusted to improve the performance of a chromatography protocol. For example, the mode and/or media used in the chromatography protocol may be adjusted to increase the efficiency and/or effectiveness of a chromatography protocol. In addition or alternatively, parameters regarding how a sample (e.g., a chromatography load) is introduced into the chromatography system (e.g., a chromatography column) may be adjusted to improve the performance of a chromatography protocol. Such parameters may include, for example, loading density, loading buffer composition, load of HMW species within a load, a dimension of a chromatography apparatus (e.g., a total volume, a bed height, an inner diameter, a membrane thickness), a media composition, a chromatography media density, one or more buffer compositions, a concentration of high molecular weight species in the load, a flow rate, a pH, a temperature, a conductivity, a salt concentration, an antibody-drug conjugate concentration of the load, and/or a drug-to-antibody ratio of the load.

Conventional methods of developing chromatography protocols, and optimizing parameters of the chromatography protocols, are time and labor intensive. The costs associated with the time and labor for developing a chromatography protocol can be prohibitive, and result in non-optimized chromatography protocols being used in the production. The use of non-optimized chromatography protocols themselves can also increase the costs associated with the production of biopharmaceutical products. Therefore, there exists a need for a high-throughput system for developing and evaluating chromatography protocols. Methods of the present disclosure may be used to generate, develop, and/or validate improved chromatography protocols.

In one aspect, the methods of the present disclosure generate an eluate in which a concentration of an impurity in an eluate of the chromatography protocol may be less than a concentration of the impurity in the load. In one aspect, the methods of the present disclosure generate a protocol for preparing a chromatography process for a load including the target molecule and impurities, and the prepared chromatography process generating an eluate in which a concentration of an impurity in the eluate may be less than a concentration of the impurity in the load.

Chromatography protocols may be implemented at various stages of the manufacture of biopharmaceutical products. In some aspects, a target molecule may undergo one or more chromatography processes. For example, a solution (i.e., the load) including the target molecule may be introduced into a chromatography system to generate an eluate including the target molecule. The eluate may have less species (e.g., less host cell proteins, less high molecular weight species, and less undesirable target molecule variants) than the solution introduced into the chromatography system. A chromatography protocol may include parameters of the chromatography operation being performed, including, but not limited to chromatography mode, column size (e.g., a total volume, a bed height, an inner diameter, a membrane thickness), a media composition, a loading density, a chromatography media density, one or more buffer compositions, a concentration of high molecular weight species in the load, a flow rate, a pH, a temperature, a conductivity, a salt concentration, an antibody-drug conjugate concentration of the load, and/or a drug-to-antibody ratio of the load.

Chromatography protocols of the present disclosure may be employed with any suitable chromatography system column configured to execute such protocol, e.g., a system including one or more chromatographic columns.

As previously noted, during chromatography operations, certain impurities may co-elute with the target molecule. Monitoring the concentration of certain impurities that end up in a drug product formulation is required to ensure that the manufactured formulation meets internal quality assurance metrics, and standards of applicable regulatory bodies.

When developing improved chromatography protocols, one or more performance criteria of potential new chromatography protocols may be compared to performance criteria of existing chromatography protocols. Performance criteria may include the concentration of one or more impurities, such as, by way of non-limiting example, a high molecular weight species composition (e.g., % HMW), a total protein content, a particle size distribution, a residual free linker-payload content, a residual organic solvent content, or an unconjugated antibody content.

In some aspects, % HMW may be measured by size-exclusion HPLC, microchip capillary electrophoresis, analytical ultracentrifugation, dynamic light scattering, mass spectrometry, SDS-PAGE analysis, analytical hydrophobic interaction chromatography, or a combination thereof. Total protein content may be measured by ultraviolet spectrophotometry or other suitable method (e.g., analytical chromatography, SDS-PAGE). In some aspects, residual free linker-payload content may be measured using analytical chromatography. Residual organic solvent content may be measured, for example, by gas chromatography-mass spectrometry. Particle size distribution may be determined, for example, by dynamic light scattering. Particle size distribution may be used to quantify an amount of aggregates or other large particle size species present in a sample.

In some aspects, an unconjugated antibody content can be determined using analytical chromatography (e.g., hydrophobic interaction chromatography). For example, a sample used as a load for a hydrophobic interaction chromatography operation, where the operation separates unconjugated antibodies from conjugated antibodies. A chromatogram may be generated during the chromatography operation where one or more peaks of the chromatogram correspond to conjugated antibodies and one or more peaks of the chromatogram correspond to unconjugated antibodies. By comparing the relative peak areas corresponding to conjugated and unconjugated antibodies, a percentage of unconjugated antibodies may be determined.

As alluded to above, some impurities have threshold values (e.g., maximum limits) that are allowed in a final drug product formulation. These threshold values can be based on internal quality assurance metrics and/or standards of applicable regulatory bodies. For example, the maximum allowable concentration of high molecular weight species (% HMW) may be approximately 9%, approximately 8%, approximately 7%, approximately 6%, approximately 5%, approximately 4%, or approximately 3%. The maximum allowable concentration of residual free linker-payload may be approximately 5 wt. %, approximately, 4 wt. %, approximately 3 wt. %, approximately 2 wt. %, approximately 1 wt. %, approximately 0.5 wt. %, or approximately 0.1 wt. %. The maximum allowable residual organic solvent concentration may depend on the solvent. In some aspect, the maximum allowable residual organic solvent concentration may be approximately 10000 parts per million (ppm), approximately 8000 ppm, approximately 5000 ppm, approximately 3000 ppm, approximately 2000 ppm, or approximately 1000 ppm. The maximum allowable unconjugated antibody concentration is approximately 15%, approximately 12%, approximately 10%, approximately 8%, or approximately 5%. An eluate including a monodisperse particle size distribution may have acceptable levels of aggregates and/or other large particle size species. In some aspects, a chromatography operation may be designed such that the eluate of the operation has a monodisperse particle size distribution, including, for example, an average particle size of less than or equal to approximately 15 nm, less than or equal to approximately 12 nm, less than or equal to approximately 10 nm, less than or equal to approximately 8 nm, or less than or equal to approximately 5 nm.

Development goals for chromatography protocols may include generating an eluate with a certain amount of high molecular weight species (e.g., an eluate having less than or equal 3% HMW). A performance criterion for a chromatography protocol may be the reduction of high molecular weight species. Reduction in HMW (% HMWΔ) for a chromatography protocol may be defined by subtracting the % HMW of the eluate from the % HMW in the load, as shown in Equation 1.

$$\% HMW Δ = \% {HMW}_{load} - \% {HMW}_{eluate} \qquad Equation 1$$

Methods of the present disclosure may include developing a chromatography protocol, wherein a % HMWΔ of the chromatography protocol is at least approximately 2%, such as, for example, at least approximately 3%, at least approximately 4%, at least approximately 5%, approximately 1% to approximately 15%, approximately 1% to approximately 12%, approximately 1% to approximately 10%, approximately 1% to approximately 8%, approximately 2% to approximately 8%, or approximately 2% to approximately 5%.

In some aspects, performance criteria may include an efficiency and/or a yield. For example, performance criteria may include a productivity and/or a total protein yield. Productivity may be defined as an amount of purified material (e.g., an amount antibody-drug conjugate eluted during a chromatography operation) generated per unit time, such as, for example, grams per hour. The total protein yield of a chromatography operation may be calculated as a ratio of a total protein concentration of the eluate (TPₑₗᵤₐₜₑ) to a total protein concentration of the load (TP_{load}), as shown in Equation 2.

$$Yield = \frac{{TP}_{eluate}}{{TP}_{load}} \qquad Equation 2$$

A protein concentration (e.g., a total protein concentration of the load or the eluate) may be measured by ultraviolet spectrophotometry, or other suitable means of determining a protein concentration. In some aspects, a total protein yield may correspond to (e.g., be equivalent to) a yield of a drug-antibody conjugate. Methods of the present disclosure may include developing a chromatography protocol, wherein a yield of a target molecule in the eluate is at least approximately 70%, such as, for example, at least approximately 75%, at least approximately 80%, at least approximately 85%, at least approximately 90%, at least approximately 95%, approximately 75% to approximately 95%, approximately 75% to approximately 90%, approximately 80% to approximately 95%, or approximately 80% to approximately 90%.

Performance criteria may include one or more metrics quantifying a yield of a drug-antibody conjugate. For example, performance criteria may include a drug-to-antibody ratio. In addition or alternatively, performance criteria may include one or more metrics of a chromatography operation that compare a drug-to-antibody ratio of a load of the operation to a drug-to-antibody ratio of an eluate of the operation. For example, performance criteria may include a difference between the load and eluate drug-to-antibody ratios or a ratio of the load and eluate drug-to-antibody ratios.

In addition or alternatively, performance criteria may include a desirability metric and/or another combination of other performance criteria. In some aspects, a desirability metric may be calculated as a composite of two or more performance criteria. Values for a performance criterion may be assigned a corresponding desirability value on a scale of 0-1, based on the favorability of the performance criterion value. For example, in aspects where a higher yield is desirable, performance criterion values for yield greater than or equal 90% may be assigned a desirability value of 1, values for yield less than or equal to 70% may be assigned a desirability value of 0, and values for yield between 70% and 90% may be assigned a value between 0 and 1 according to a function (e.g., a linear function).

The desirability metric may be calculated as a composite (e.g., an average) of the component desirability values for the corresponding performance criteria. The desirability metric may be structured such that each performance criterion of the composite is equally weighted. In some aspects, the desirability metric may be structured such that each performance criterion of the composite is assigned a weight that increases or decreases its contribution to the desirability metric. For example, a desirability metric may be structured such that desirability values corresponding to % HMW or drug-to-antibody ratio performance criteria are more heavily weighted than other performance criteria. As another example, desirability values corresponding to % HMW or drug-to-antibody ratio could be given the greatest weight, desirability values corresponding to particle distribution could be given the second greatest weight, and desirability values corresponding to yield could be give the least weight.

In aspects where the target molecule is an antibody-drug conjugate, the development of chromatography protocols (e.g., HIC protocols) may present additional challenges. For example, antibody-drug conjugates may include increased heterogeneity, compared to other biopharmaceutical products, due to the multiplicity of sites available (e.g., reactive lysine attachment sites) for conjugation on the antibody. The variations in conjugation of drug to antibody may result in the generation of many different species during production of the biopharmaceutical product. The various species of antibody-drug conjugates may have different drug-to-antibody ratios, hydrophobicity, and aggregation propensity. Therefore, in some embodiments, impurities such as aggregates may have similar surface hydrophobicities, and different drug-to-antibody ratios, compared to the target molecule. Accordingly, there exists a need for improved chromatography protocols that are designed for target molecules including an antibody-drug conjugate. The high-throughput methods of developing and evaluating chromatography protocols described herein may be used to develop chromatography protocols for an antibody-drug conjugate as a target molecule.

Systems and methods disclosed herein may provide an improved development flow for chromatography protocols. The improved development flow may result in improved chromatography protocols. In one or more embodiments, the systems and methods of the present disclosure allow for the development of predictive models that enable the high-throughput evaluation of chromatography protocols. Predictive models may be generated that can quantify a one or more performance criteria based on a set of chromatography parameters (e.g., loading parameters).

Certain chromatography modes or chromatography media types may be chosen for a particular operation. For example, in chromatography operations that require a reduction in high molecular weight species and/or aggregates, hydrophobic interaction chromatography may be utilized to separate high molecular weight species and/or aggregates from a target molecule (e.g., an antibody-drug conjugate). After a mode of chromatography is selected based on the goals of the chromatography operation, development of a chromatography protocol may include determining a type of chromatography media for use in the chromatography protocol.

Throughout the present disclosure, references is made to exemplary chromatography protocols, including hydrophobic interaction chromatography (HIC) protocols. It is noted that while describing systems and methods of the present disclosure, reference may be made to one or more HIC protocols; such systems and methods are not necessarily limited to HIC, and may be applicable to various modes of chromatography (e.g., affinity chromatography (e.g., Protein A or Protein L), ion-exchange chromatography, size-exclusion chromatography, reverse phase chromatography, fast protein liquid chromatography, high-performance liquid chromatography, countercurrent chromatography, periodic counter-current chromatography, chiral chromatography, and/or mixed-mode chromatography).

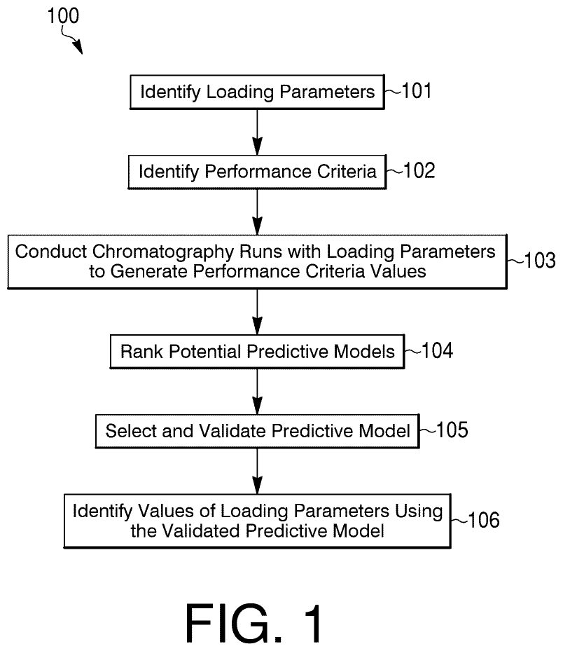

Referring to FIG. 1, a method 100 of developing a chromatography protocol may include identifying loading parameters (step 101), identifying performance criteria (step 102), conducting chromatography runs with loading parameters to generate performance criteria values (step 103), rank potential predictive models (step 104), select and validate a predictive model (step 105), and using the validated predictive model, identify values of loading parameters for the chromatography protocol (step 106).

Identifying loading parameters may include identifying, selecting, and/or determining parameters of a chromatography protocol that will be varied during the development of the protocol. For example, loading parameters may include “input variables” or aspects of the chromatography protocol that may be adjusted, varied, controlled, while the effect of the loading parameters on performance criteria are monitored and/or recorded. As noted above, examples of loading parameters include, but are not limited to, chromatography mode, column size (e.g., a total volume (e.g., a column volume or a membrane volume), a bed height, a membrane thickness, an inner diameter, etc.), a media composition, a loading density, one or more buffer compositions, a concentration of high molecular weight species in the load, and a flow rate.

As noted above, various modes of chromatography may include, but are not limited to, hydrophobic interaction chromatography, affinity chromatography (e.g., Protein A or Protein L), ion-exchange chromatography, size-exclusion chromatography, reverse phase chromatography, fast protein liquid chromatography, high-performance liquid chromatography, countercurrent chromatography, periodic counter-current chromatography, chiral chromatography, and/or mixed-mode chromatography. The selection of a specific chromatography mode may take into account various considerations, including the type of load, preferred means of separation, the scale of the procedure, etc. The selected chromatography mode may then, in turn, affect chromatography operational parameters including, but not limited to, the type of chromatography media, i.e., stationary phases, chromatography mobile phases, the buffer compositions, etc.

Column dimensions may include a total volume (e.g., a column volume or a membrane volume), a bed height, a membrane thickness, and/or an inner diameter. Exemplary column volumes may be approximately 0.2 milliliters (mL) to approximately 4600 mL. Exemplary bed heights may be approximately 1 centimeter (cm) to approximately 30 cm. Exemplary inner diameters may be approximately 1 cm to approximately 14 cm. Exemplary membrane volumes may be approximately 0.08 mL to approximately 5 liters. Exemplary membrane thicknesses may be approximately 4 millimeters (mm) to approximately 8 mm.

A chromatographic column may comprise a type of chromatography media. For example, chromatographic columns may include amino acid media, ligand-specific media, immunoaffinity media, ion affinity media, hydrophobic interaction media, and/or charged media. The media may be in the form of resin, beads, particles bound in a packed bed column format, a membrane, or in any format that can accommodate a mixture or other liquid comprising biopharmaceutical products. The media may include a support structure such as, for example, agarose beads (e.g., sepharose), silica beads, cellulosic membranes, cellulosic beads, hydrophilic polymer beads, or other compactable synthetic structure. Selection of chromatography media may be based on a number of different factors including, for example, the mode of chromatography and the analyte type, i.e., the species to be separated during chromatography. A chromatography media may be selected to optimize interactions between the media and the analyte.

Loading density may be quantified in terms of mass (e.g., grams) of antibody-drug conjugate loaded per volume of chromatography apparatus (e.g., column volume or membrane volume) that the conjugate was loaded onto. Chromatography protocols with higher loading density may result in greater interaction of the media with the mobile phase, compared to chromatography protocols using a lower loading density. Increased loading density may result in improved reduction of high-molecular weight species. Chromatography protocols using an increased loading density can also result in reduced yield, compared to chromatography protocols using a lower loading density.

In some aspects, a loading density of a chromatography operation may be approximately 10 grams per liter (g/L) to approximately 40 g/L, such as, for example, approximately 20 g/L to approximately 30 g/L, approximately 10 g/L to approximately 25 g/L, approximately 15 g/L to approximately 30 g/L, approximately 15 g/L to approximately 25 g/L, approximately 25 g/L to approximately 30 g/L, or approximately 20 g/L to approximately 25 g/L.

Loading parameters may include the composition of one or more buffers of the chromatography protocol, such as, for example, the composition of a loading buffer, or other buffer that constitutes a mobile phase of the chromatography protocol. The composition of the one or more buffers may affect how components of the load interaction with each other and/or the chromatography media. Loading parameters related to buffer composition may include the presence and/or concentration of one or more salts or other compounds in the buffer. In addition or alternatively, loading parameters related to buffer composition may include a pH of the buffer.

For example, one or more buffers of a chromatography protocol may include one or more salts (e.g., a citrate salt). Loading parameters may be binary parameters referring to the presence of the one or more salts. In some aspects, loading parameters may include the concentration of the one or more salts (e.g., expressed in as a millimolar [mM]concentration). For example, a chromatography protocol may include a citrate concentration of a loading buffer, such as, for example, a citrate concentration of approximately 5 mM to approximately 200 mM, 5 mM to approximately 150 mM, 5 mM to approximately 100 mM, 25 mM to approximately 200 mM, 50 mM to approximately 200 mM, approximately 100 mM to approximately 200 mM, approximately 100 mM to approximately 150 mM, approximately 150 mM to approximately 200 mM, approximately 110 mM to approximately 175 mM, approximately 50 mM to approximately 125 mM, approximately 110 mM to approximately 200 mM, approximately 140 mM to approximately 155 mM, approximately 110 mM to approximately 155 mM, or approximately 140 mM to approximately 175 mM.

Loading parameters may include a high molecular weight species content of the chromatography load. For example, loading parameters may include % HMW of the load. The % HMW of the load may be approximately 3% to approximately 20%, such as for example, approximately 3% to approximately 15%, approximately 3% to approximately 10%, approximately 5% to approximately 20%, approximately 5% to approximately 15%, approximately 5% to approximately 10%, approximately 7% to approximately 20%, approximately 7% to approximately 15%, or approximately 7% to approximately 10%.

Loading parameters may also include one or more other properties of the load, such as viscosity and/or density. In some embodiments, properties of the load (e.g., high molecular weight species content, viscosity, and/or density) may be adjusting during production, or after production and prior to execution of the chromatography protocol.

Referring again to FIG. 1, a method 100 of developing a chromatography protocol may include identifying performance criteria (step 102). Performance criteria may include metrics quantifying the performance, efficiency, and/or effectiveness of the chromatography protocol. Performance criteria may include “output variables” or aspects of the chromatography protocol that depend on the values chosen for loading parameters. Identifying performance criteria may include identifying, selecting, and/or determining performance of a chromatography protocol that will be used to evaluate potential chromatography protocols during development.

Examples of performance criteria include, but are not limited to, a high molecular weight species composition (e.g., % HMW), a reduction in high molecular weight species content (e.g., % HMWΔ), a productivity, a total protein yield, a drug-to-antibody ratio, a difference of drug-to-antibody ratios, a ratio of drug-to-antibody ratios, a total protein content, a particle size distribution, a residual free linker-payload content, a residual organic solvent content, an unconjugated antibody content, or a desirability metric.

Referring again to FIG. 1, a method 100 of developing a chromatography protocol may include conducting chromatography runs to generate performance criteria values (step 103). Chromatography runs may be conducted with the identified loading parameters being varied. Other loading parameters may be held constant while the identified loading parameters are varied. For example, if a flow rate and loading density were identified as loading parameters, all other parameters of the chromatography protocol (e.g., chromatography mode, column size, media composition, buffer compositions, and concentration of high molecular weight species in the load) may be held constant while chromatography runs are conducted. Continuing on the example where flow rate and loading density were identified as loading parameters, conducting chromatography runs may include conducting chromatography runs according to chromatography protocols with varying flow rates and media densities. Each chromatography run conducted may generate a value for each identified performance criteria. For example, when four performance criteria are identified, each chromatography run may generate four values, one for each performance criterion.

Referring again to FIG. 1, a method 100 of developing a chromatography protocol may include ranking potential predictive models (step 104). As described in greater detail herein, a domain of predictive models may generated, and/or a pool of candidate predictive models may be identified. Each predictive model may include a mathematical relation of the identified loading parameters to the identified performance criteria. For example, a predictive model may be able to predict values of identified performance criteria based on a given set of values for identified loading parameters. In aspects where multiple performance criteria are identified, potential predictive models may include a function that corresponds to each identified performance criterion.

As described in greater detail herein, potential predictive models may be used to generate projected performance criteria values. The projected performance criteria values may be compared to the performance criteria values generated from the chromatography runs (step 103). The comparison of projected performance criteria values to the performance criteria values generated from the chromatography runs may result in a quantification for each predictive model corresponding to the correlation of the predictive model to the data generated from the chromatography runs. For example, a coefficient of determination (R²) may be assigned to each potential predictive model, based on the correlation of the projected performance criteria values generated from that model to the performance criteria values generated from the chromatography runs. Each potential predictive model may be ranked based on the quantification (e.g., coefficient of determination) of the predictive model's correlation to the data generated from the chromatography runs.

Referring again to FIG. 1, after the predictive models are ranked according to the correlation between the projected performance criteria values and the performance criteria generated from the chromatography runs, a predictive model may be selected and validated (steps 105). For example, in one or more embodiments, the predictive model with the highest rank may be selected. After a predictive model is selected, the predictive model may be validated via additional chromatography runs. Validating a selected predictive model may include further studies to ensure that the predictive model correlates to observed phenomena over a large range of loading parameter values.

Further, chromatography protocols developed by methods of the present disclosure may be validated with further studies, to ensure that biopharmaceutical products prepared using the developed chromatography protocol meet internal quality assurance metrics and standards of applicable regulatory bodies. The further studies may include characterization of protein concentration, high molecular weight species content, drug-to-antibody ratio, drug-load distribution (e.g., using mass spectrometry), and/or particle size distribution (e.g., using dynamic light scattering). In addition or alternatively, the further studies may include peptide mapping, binding assays, and other bioassays of the species within the eluate generated by the chromatography protocol. In some aspects, validating a chromatography protocol may include conducting one or more in vivo studies. For example, in vivo studies using suitable analog organisms may be conducted to determine pharmacokinetics, pharmacodynamics, efficacy, and/or toxicity of species generated using the developed chromatography protocol.

Still referring to FIG. 1, after the selected predictive model is validated, the predictive model may be used to evaluate thousands of chromatography protocols in a high-throughput manner. The rate at which chromatography protocols can be evaluated with the predictive model may enable solving for optimum loading parameter values, thereby determining an improved chromatography protocol. For example, the validated predictive model may be utilized to solve for values of loading parameters that may provide optimum values of one or more performance criteria, such as, for example, a desirability metric.

As more predictive models are generated and the library of chromatography run data grows, more accurate predictive models will be generated for each identified performance criteria. All performance criteria for all loading parameters may be assessed in the aforementioned high-throughput manner, to determine which loading parameters result in sufficient performance criteria. Advantageously, the high-throughput manner of evaluating chromatography protocols may reduce the time, labor, and costs associated with developing chromatography protocols suitable for use in the production of biopharmaceutical products.

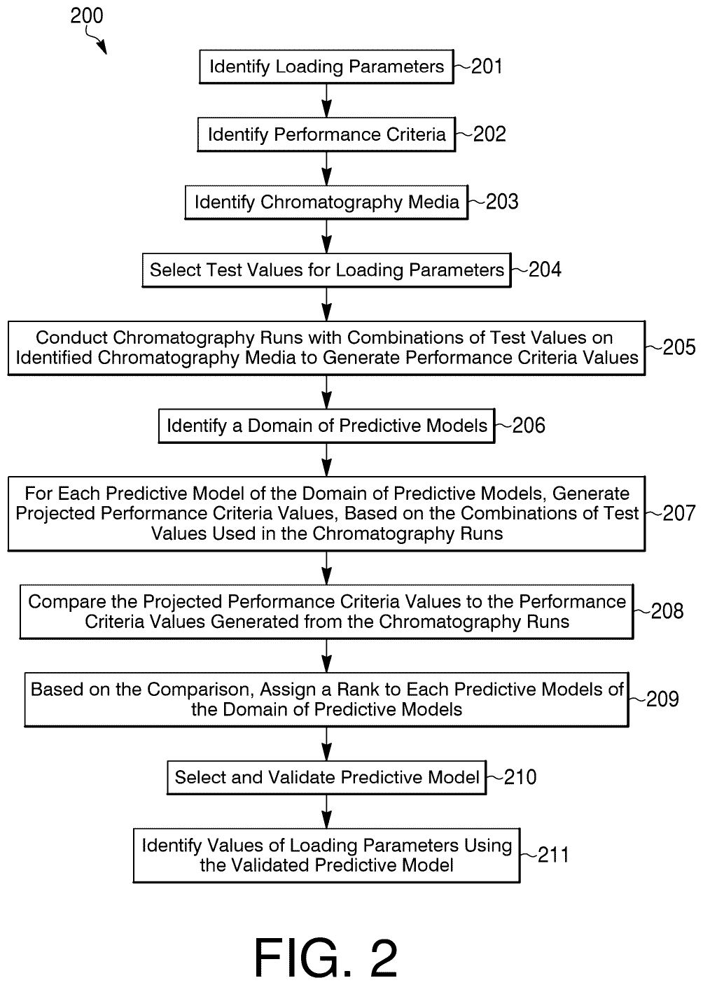

Referring to FIG. 2, a method 200 of developing a chromatography protocol may include identifying loading parameters (step 201), identifying performance criteria (step 202), identifying chromatography media (step 203), selecting test values for loading parameters (step 204), conducting chromatography runs with loading parameters test values on chromatography media to generate performance criteria values (step 205), identifying a domain of predictive models (step 206), for each predictive model of the domain of predictive models, generating projected performance criteria values, based on the combinations of test values used in the chromatography runs (step 207), comparing the projected performance criteria values to the performance criteria values generated from the chromatography runs (step 208), based on the comparison, assigning a rank to each predictive models of the domain of predictive models (step 209), selecting and validating a predictive model (step 210), and identifying values of loading parameters using the validated predictive model (step 211).

Some steps of method 200, such as, for example, identifying loading parameters (step 201), identifying performance criteria (step 202), conducting chromatography runs with loading parameters test values on chromatography media to generate performance criteria values (step 205), assigning a rank to each predictive models of the domain of predictive models (step 209), selecting and validating a predictive model (step 210), and identifying values of loading parameters using the validated predictive model (step 211), may be similar to steps of method 100 for developing chromatography protocols, as described above. In some aspects, method 200 of developing a chromatography protocol may include additional steps that are not required in method 100.

Still referring to FIG. 2, a method 200 of developing a chromatography protocol may further include identifying chromatography media (step 203). For example, for a given mode of chromatography, there may be several candidate chromatography media that could be used in a chromatography protocol. Chromatography media may include amino acid media, ligand-specific media, immunoaffinity media, ion affinity media, hydrophobic interaction media, and/or charged media. Chromatography media may be in the form of resin, beads, particles bound in a packed bed column format, a membrane, or in any format that can accommodate a mixture or other liquid comprising biopharmaceutical products. Chromatography media may include a support structure such as, for example, agarose beads (e.g., sepharose), silica beads, cellulosic membranes, cellulosic beads, hydrophilic polymer beads, or other compactable synthetic structure.

The suitability of a given media for use in a chromatography protocol may depend on the identity of the target molecule and other species within the mixture that constitutes the chromatography load. In some aspects, other factors may impact the suitability of a given media for use in a chromatography protocol, such as, for example, dimensions of the column, or other loading parameters of the chromatography protocol. Accordingly, certain chromatography media exhibit superior performance (e.g., measured by one or more performance criteria) for some chromatography protocols, compared to other chromatography media. Any changes to a target molecule or chromatography protocol may result in changes in the suitability of a selected chromatography media. Therefore, in some aspects, a method 200 of developing a chromatography protocol may include identifying or determining a chromatography media suitable for the chromatography protocol.

Identifying chromatography media may include performing chromatography runs with different types of chromatography media, which may generate one or more performance criteria corresponding to each type of chromatography media used in the chromatography runs. When identifying chromatography media, the chromatography runs may use identical chromatography protocols, with the exception of the chromatography media used, to better understand the impact of the chromatography media on the performance criteria. Identifying chromatography media may further include monitoring the performance criteria corresponding to the different types of chromatography media, and ranking the chromatography media according to their corresponding performance criteria. A chromatography media with the highest ranking may be selected for use in a chromatography protocol.

As an illustrative and non-limiting example, identifying a chromatography media may include performing a chromatography run using eight different types of chromatography media, for a total of eight chromatography runs. Each chromatography run may generate values of one or more performance criteria (e.g., a desirability metric) corresponding to each type of chromatography media. The eight media may be ranked according to their corresponding performance criteria, and the media with the highest ranking (e.g., the greatest desirability metric) may be selected. In some aspects, multiple chromatography runs may be performed for each type of chromatography media. The values of performance criteria generated during the multiple runs for one type of chromatography media may be averaged to generate a performance criteria value corresponding to the type of chromatography media.

As described above in relation to method 100, chromatography runs may be conducted with combinations of test values to generate performance criteria. Referring to FIG. 2, a method 200 of developing a chromatography protocol may include selecting test values of loading parameters for chromatography runs (step 204). In one or more embodiments, test values are selected for each loading parameter. Combinations of test values may include every possible combination of the test values selected for each loading parameters. For example, if loading density, a loading buffer sulfate concentration, and a high molecular weight species content are selected as loading parameters, selecting test values may include selecting test values for loading density (e.g., 20 g/L, 35 g/L, and 50 g/L), a loading buffer citrate concentration (e.g., 130 mM, 150 mM, and 170 mM), and a high molecular species content (e.g., 12% HMW, 8% HMW, and 4% HMW). In this example, combinations of test values including every possible combination of test values selected for each loading parameter may include the combinations of test values shown below in Table 1.

TABLE 1

| | First Loading | | Second Loading | | Third Loading |
|---|---|---|---|---|---|
| Chromatography | Parameter Test | | Parameter Test | | Parameter Test |
| Run No. | Values | | Values | | Values |
| 1 | 20 | g/L | 130 | mM | 12% |
| 2 | 20 | g/L | 130 | mM | 8% |
| 3 | 20 | g/L | 130 | mM | 4% |
| 4 | 20 | g/L | 150 | mM | 12% |
| 5 | 20 | g/L | 150 | mM | 8% |
| 6 | 20 | g/L | 150 | mM | 4% |
| 7 | 20 | g/L | 170 | mM | 12% |
| 8 | 20 | g/L | 170 | mM | 8% |
| 9 | 20 | g/L | 170 | mM | 4% |
| 10 | 35 | g/L | 130 | mM | 12% |
| 11 | 35 | g/L | 130 | mM | 8% |
| 12 | 35 | g/L | 130 | mM | 4% |
| 13 | 35 | g/L | 150 | mM | 12% |
| 14 | 35 | g/L | 150 | mM | 8% |
| 15 | 35 | g/L | 150 | mM | 4% |
| 16 | 35 | g/L | 170 | mM | 12% |
| 17 | 35 | g/L | 170 | mM | 8% |
| 18 | 35 | g/L | 170 | mM | 4% |
| 19 | 50 | g/L | 130 | mM | 12% |
| 20 | 50 | g/L | 130 | mM | 8% |
| 21 | 50 | g/L | 130 | mM | 4% |
| 22 | 50 | g/L | 150 | mM | 12% |
| 23 | 50 | g/L | 150 | mM | 8% |
| 24 | 50 | g/L | 150 | mM | 4% |
| 25 | 50 | g/L | 170 | mM | 12% |
| 26 | 50 | g/L | 170 | mM | 8% |
| 27 | 50 | g/L | 170 | mM | 4% |

Selecting test values for loading parameters may include selecting two or more test values for each loading parameter, for example, selecting test values may include selecting two, three, four, five, six, seven, eight, or nine test values for each loading parameter. Selecting test values for loading parameters may include selecting less than or equal to ten test values, less than or equal to eight test values, less than or equal to six test values, or less than or equal to five test values. In some aspects, the number of test values selected for one loading parameter may be the same as the number of test values selected for each other loading parameter. In other aspects, the number of test values selected for a first loading parameter may be different than the number of test values selected for a second loading parameter. For example, more test values may be selected for loading parameters that are thought to create more variability and/or have a larger operational range. Including more test values for a loading parameter may increase the amount of time required for chromatography runs based on the test value combinations, but may provide a more robust data set to use in developing a predictive model.

In some aspects, selecting test values of loading parameters for chromatography runs may include selecting combinations of test values may be selected. Stated differently, instead of individually selecting test values for each loading parameter, and conducting chromatography runs with every possible combination of selected test values, combinations of test values of loading parameters may be selected. For example, if loading density, a loading buffer sulfate concentration, and a high molecular weight species content are selected as loading parameters, selecting test values may include selecting the combinations of test values shown in Table 2.

TABLE 2

| | First Loading | | Second Loading | | Third Loading |
|---|---|---|---|---|---|
| Chromatography | Parameter Test | | Parameter Test | | Parameter Test |
| Run No. | Values | | Values | | Values |
| 1 | 15 | g/L | 130 | mM | 12% |
| 2 | 15 | g/L | 160 | mM | 10% |
| 3 | 15 | g/L | 190 | mM | 8% |
| 4 | 20 | g/L | 135 | mM | 10% |
| 5 | 20 | g/L | 145 | mM | 8% |
| 6 | 20 | g/L | 195 | mM | 6% |
| 7 | 30 | g/L | 130 | mM | 12% |
| 8 | 30 | g/L | 150 | mM | 9% |
| 9 | 30 | g/L | 170 | mM | 6% |
| 10 | 40 | g/L | 140 | mM | 10% |
| 11 | 40 | g/L | 160 | mM | 7% |
| 12 | 40 | g/L | 180 | mM | 4% |
| 13 | 50 | g/L | 135 | mM | 12% |
| 14 | 50 | g/L | 145 | mM | 8% |
| 15 | 50 | g/L | 165 | mM | 4% |
| 16 | 55 | g/L | 130 | mM | 10% |
| 17 | 55 | g/L | 160 | mM | 9% |
| 18 | 55 | g/L | 180 | mM | 8% |

Advantageously, by selecting combinations of test values instead of using every combination of selected test values, more test values may be tested for each loading parameter, while using less chromatography runs. Utilization of more test values during chromatography runs may improve the robustness of predictive models developed using the methods described herein. Additionally, conducting chromatography runs can be time and labor intensive, which as described previously, can increase the associated costs. Therefore, reducing the number of chromatography runs of step 205 can reduce the total cost associated with methods 200 of developing chromatography protocols.

Referring again to FIG. 2, a method of developing a chromatography protocol may include conducting chromatography runs with combinations of test values on identified chromatography media to generate performance criteria values (step 205). For example, one chromatography run may be conducted for each selected combination of test values, thereby generating a value for each identified performance criterion that corresponds to the selected combination of test values. In some embodiments, multiple chromatography runs (e.g., 3 or more chromatography runs) may be conducted for each selected combination of test values. The performance criteria generated from the multiple chromatography runs may be averaged to generate performance criteria values. In some embodiments, the chromatography runs of step 205 may be conducted using the chromatography media identified in step 203.

Referring to FIG. 2, a method 200 of developing a chromatography protocol may further include identifying a domain of predictive models. Identifying a domain of predictive models may include identifying a domain of predictive models for each identified performance criterion. For each identified performance criterion, a predictive model may include a mathematical relationship between one or more of the identified loading parameters and the identified performance criterion. For example, if two performance criteria are identified, step 206 may include identifying a first domain of predictive models that relate one or more loading parameters to a first performance criterion and identifying a second domain of predictive models that relate one or more loading parameters to a second performance criterion.

The domain of predictive models may include univariate models, bivariate models, trivariate models, or other multivariate models. For example, products, quotients, exponents, and other multivariate relationships of loading parameters may be considered. The domain of potential models may also include known mechanistic or empirical relationships.

In some aspects, the structure of the predictive models may be determined by the number of identified loading parameters such that the number of independent variables in the predictive model is less than or equal to the number of identified loading parameters. For example, in embodiments where two loading parameters are identified, potential predictive models may include univariate and bivariate models. In aspects where three loading parameters are identified, potential predictive models may include univariate, bivariate, and trivariate models.

The domain of predictive models may include tens of thousands of models, such as, for example, greater than 50,000 potential predictive models. In some aspects, additional steps may be used to reduce the number of potential predictive models in the domain of predictive models. For example, models with duplicate parameters may be removed, such that the domain of predictive models does not include duplicative models. In developing algebraic expressions relating evaluation criterion to loading parameters, equivalent expressions may be created. These equivalent expressions may functionally be duplicates that can be removed from the domain.

In some aspects, the variance inflation factor (VIF) of each potential predictive model may be calculated and models with a VIF greater than or equal to a collinearity threshold may be excluded from the domain of predictive models. In some aspects, the collinearity threshold is four or less, such as, for example, two, three, or four. After models with duplicate parameters and models with a VIF greater than or equal to a collinearity threshold are removed, the domain of predictive models may include hundreds of models. For example, for each identified performance criterion, the remaining domain of predictive models may include less than or equal to 500 models.

Referring to FIG. 2, a method 200 of developing a chromatography protocol may include generating projected performance criteria values, for each predictive model of the domain of predictive models (step 207). The projected performance criteria values may be generated by using the combinations of test values, such that for each predictive model, one projected performance criteria value is generated that corresponds to each combination of test values. For example, if the chromatography runs of step 205 were performed with 27 combinations of test values, each predictive model of the domain of predictive models may be used to generate 27 projected performance criteria values. In this example, if two performance criteria are identified, a first domain of predictive models for the first performance criterion includes 500 models, and a second domain of predictive models for a second performance criterion includes 600 models, generating projected performance criteria values may include generating 29,700 projected performance criteria values.

Referring to FIG. 2, a method 200 of developing a chromatography protocol may include comparing the projected performance criteria values to the performance criteria values generated from the chromatography runs of step 205 (step 208). Comparing projected performance criteria values to the performance criteria values generated from the chromatography runs may include generating a statistical metric for each predictive model of the domain of predictive models that relates to how well the predictive model correlates to observed phenomena. For example, based on the comparison of the projected performance criteria values to the performance criteria values generated from the chromatography runs, a coefficient of determination (R²) may be calculated for each predictive model of the domain of predictive models.

Still referring to FIG. 2, a method 200 of developing a chromatography protocol may include assigning a rank to each predictive model of the domain of predictive models, based on the comparison of projected performance criteria values to the performance criteria values (step 209). For example, the domain of predictive models may be ranked based on the statistical metric that relates to the correlation of the predictive models within the domain. In some aspects, a coefficient of determination is calculated for each predictive model, and the domain is ranked in descending order based on the coefficients of determination.

As described above in relation to method 100, method 200 of developing a chromatography protocol may include selecting and validating a predictive model (step 210) and identifying values of loading parameters for the chromatography protocol using the validated predictive model (step 211).

### EXAMPLES

### Example 1

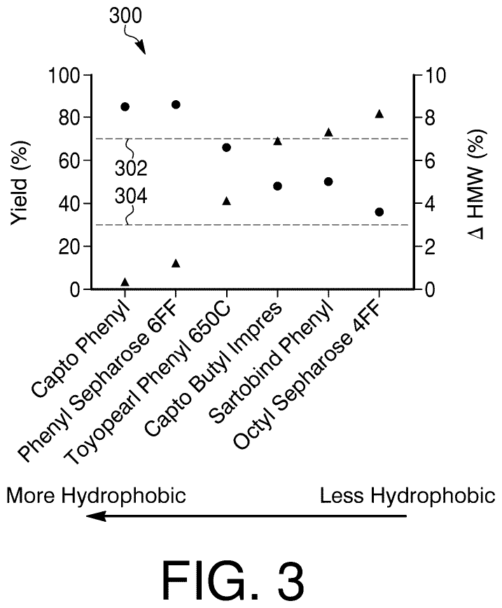

Six chromatography runs were performed using six different hydrophobic interaction chromatography (HIC) media, according to a chromatography protocol for a target molecule including an antibody-drug conjugate. Specifically, the antibody drug conjugate includes an IgG4 antibody attached via random lysine conjugation to a cleavable maytansinoid (M114). The chromatography protocol specified a loading buffer including 175 mM citrate, at a pH of 8. The chromatography protocol further specified a loading density of 20 g/L and a load % HMW of approximately 8% to approximately 9.5%. The six HIC media included capto phenyl, phenyl sepharose 6 fast flow, toyopearl phenyl-650C, capto butyl impres, sartobind phenyl, and octyl sepharose 4 fast flow, each of which has a different hydrophobicity. The relative hydrophobicities of these media are shown in FIG. 3.

For each of the six chromatography runs, two performance criteria were recorded: yield of the target molecule and high molecular weight species reduction (% HMWΔ). These performance criteria were plotted and are shown in Plot 300 of FIG. 3. Plot 300 includes lines 302 and 304 which may identify desired performance criteria values of the chromatography protocol being developed. For example, line 302 corresponds to a yield of approximately 70%, and line 304 corresponds to a % HMWΔ of approximately 3%. As shown in Plot 300, yield was inversely proportional to media hydrophobicity and % HMWΔ was inversely proportional to media hydrophobicity.

Additionally, the mass balances of total protein, high molecular weight species, and free linker-payload associated with the six chromatography runs were calculated according to Equation 3, shown below.

$$Equation 3$$

$$Mass Balance = ( \frac{Total Mass in Eluate and Strip Fraction}{Total Mass in Load} ) \times 100$$

A summary of the calculated total protein mass balances, high molecular weight species mass balances, and free linker-payload mass balances is shown below in Table 3.

TABLE 3

| | Mass Balance | | |
|---|---|---|---|
| | | High Molecular | |
| | Total Protein | Weight Species | Free Linker- |
| Media | (%) | (%) | Payload (%) |
| Capto Phenyl | 47 | 33 | 41 |
| Phenyl Sepharose 6FF | 54 | 40 | 49 |
| Toyopearl Phenyl 650C | 93 | 73 | 86 |
| Capto Butyl ImpRes | 80 | 44 | 82 |
| Sartobind Phenyl | 109 | 103 | 109 |
| Octyl Sepharose 4FF | 95 | 80 | 97 |

Based on the data in Plot 300 and Table 3, sartobind phenyl media was shown to be a preferred media that may provide optimal balancing between HMW removal, target molecule yield, and recovery of protein, aggregates, and linker-payload. Accordingly, sartobind phenyl may be a suitable starting point when developing new chromatography protocols, e.g., HIC protocols, and particularly in the instances of a target molecule including a lysine conjugated antibody-drug conjugate.

### Example 2

In one example of developing a chromatography protocol, loading density, loading buffer citrate concentration, and load % HMW were identified as three loading parameters. Yield and eluate % HMW were identified as performance criteria. Additionally a desirability metric was constructed that was a composite of yield and eluate % HMW, where yield and eluate % HMW were equally weighted. Test values for the loading parameters were selected, and chromatography runs were conducted with the combinations of test values to generate performance criteria values including yield values and eluate % HMW values. Desirability metric values were calculated based on the generated yield and eluate % HMW values. The chromatography runs were conducted on a chromatography system comprising a column including sartobind phenyl hydrophobic interaction media.

A first domain of predictive models was identified, where each model included a mathematical relation of yield as a function of one or more of loading density, loading buffer citrate concentration, and load % HMW. A second domain of predictive models was identified, where each model included a mathematical relation of eluate % HMW, as function of one or more of loading density, loading buffer citrate concentration, and load % HMW.

For each model of the first domain of predictive models, projected yields were generated corresponding to each of the test value combinations. The projected yields were compared to the generated yield values, and a coefficient of determination was calculated for each predictive model of the first domain of predictive models, based on this comparison. The predictive models of the first domain of predictive models were ranked in descending order according to their coefficient of determination, and the predictive model with the highest coefficient of determination was selected as the first predictive model.

The first predictive model can be described according to Equation 4, shown below. PGP-27 E

$$Yield = Ax + By + Cz + {D ( y + E )}^{2} ( z + F ) + G ( z + F ) + H \qquad Equation 4$$

Referring to Equation 4, x represents the loading density in grams per liter, y represents the millimolar citrate concentration of the loading buffer, z represents the % HMW of the load, and A, B, C, D, E, F, G, and H are constants. In some aspects, the first predictive model may include a value of A of approximately 0.01 L/g to approximately 1.00 L/g, such as, for example, approximately 0.354 L/g. The first predictive model may include a value of B of approximately −1.00 mL/mole to approximately −0.01 mL/mole, such as, for example, approximately −0.155 mL/mole. The first predictive model may include a value of C of approximately −10.0 to approximately −0.01, such as, for example, approximately −2.04. The first predictive model may include a value of D of approximately −0.1 (mL/mole)² to approximately 0.1 (mL/mole)², such as, for example, approximately −0.005 (mL/mole)². The first predictive model may include a value of E of approximately −500 to approximately 500, such as, for example, approximately −150. The first predictive model may include a value of F of approximately −15 to approximately −0.01, such as, for example, approximately −8.814. The first predictive model may include a value of G of approximately 0.01 to approximately 1.0, such as, for example, approximately 0.473. The first predictive model may include a value of H of approximately 10 to approximately 200, such as, for example, approximately 102.326.

For each predictive model of the second domain of predictive models, projected eluate % HMW values were generated corresponding to each of the test value combinations. The projected eluate % HMW values were compared to the generated eluate % HMW values, and a coefficient of determination was calculated for each predictive model of the second domain of predictive models, based on this comparison. The predictive models of the second domain of predictive models were ranked in descending order according to their coefficient of determination, and the predictive model with the highest coefficient of determination was selected as the second predictive model.

The second predictive model can be described according to Equation 5, shown below.

$$Equation 5$$

$$\% {HMW}_{Eluate} = Ax + By + Cz + {D ( y + E )}^{2} ( z + F ) + G ( z + F ) + H$$

Referring to Equation 5, x represents the loading density in grams per liter, y represents the millimolar citrate concentration of the loading buffer, z represents the % HMW of the load, and A, B, C, D, E, F, G, and H are constants. In some aspects, the second predictive model may include a value of A of approximately 0.01 L/g to approximately 1.00 L/g, such as, for example, approximately 0.078 L/g. The second predictive model may include a value of B of approximately −1.00 mL/mole to approximately 1.00 mL/mole, such as, for example, approximately −0.025 mL/mole. The second predictive model may include a value of C of approximately 0.01 to approximately 1.0, such as, for example, approximately 0.433. The second predictive model may include a value of D of approximately −0.1 (L/g)² to approximately 0.1 (L/g)², such as, for example, approximately −0.001 (L/g)². The second predictive model may include a value of E of approximately −200 to approximately 200, such as, for example, approximately −33.75. The second predictive model may include a value of F of approximately −0.1 L/g to approximately 0.1 L/g, such as, for example, approximately 0.008 L/g. The second predictive model may include a value of G of approximately −15.0 to approximately 15.0, such as, for example, approximately −8.814. The second predictive model may include a value of H of approximately 0.01 to approximately 10.0, such as, for example, approximately 1.985.

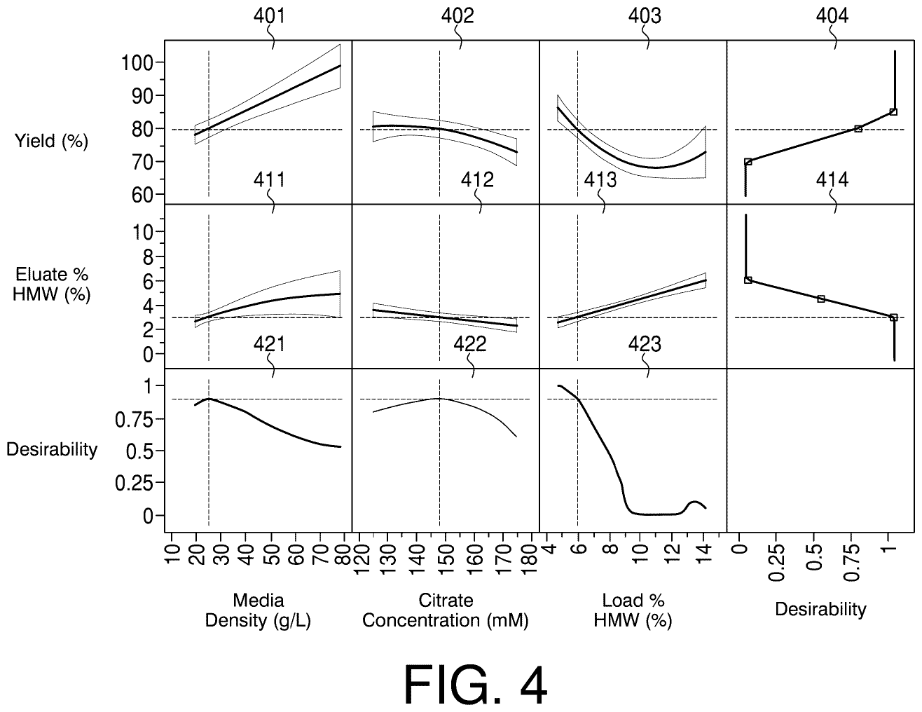

The first and second predictive models were dynamically plotted together in order to visualize the effect of adjusting loading parameters on the performance criteria. The desirability metric, which combines values for other performance criteria, was also plotted. The dynamic plot based on the first and second predictive models displays projected performance criteria based on a selection of loading parameters. The dynamic plot also displays how adjustments to the loading parameters affect performance criteria. A static screenshot of the dynamic plot, with a loading density of approximately 25 g/L, loading buffer citrate concentration of approximately 148 mM, and a load % HMW of approximately 6%, selected as loading parameters, is shown in FIG. 4. In the dynamic plot, if one of loading parameter selections change, the curves displayed in plots 401, 402, 404, 411, 412, 413, 414, 421, 422, and 423 will also change.

Referring to FIG. 4, the dynamic plot includes a first set of plots 401, 402, 403 that are generated using the first predictive model, a second set of plots 411, 412, 413 that are generated using the second predictive model. The dynamic plot may also include a third set of plots 421, 422, 423 that are generated using the first and second predictive models, combined according to a desirability metric. The dynamic plot may also include plots 404, 414 representing desirability values associated with each performance criterion, as part of a desirability metric. For example, as described herein, values associated with a performance criterion (e.g., yield, % HMW) may be assigned a desirability value on a scale from 0 to 1. The desirability values of each performance criterion may be combined into a desirability metric, based on weights associated with each performance criterion. Plot 404 shows desirability values associated with different yield values. Plot 414 shows desirability values associated with different eluate % HMW values. In the example shown in FIG. 4, the desirability values associated with yield and eluate % HMW are assigned the same weight and used to generate the desirability metric that is plotted in plots 421, 422, and 423.

The vertical dashed lines shown in each plot 401, 402, 403, 411, 412, 413, 421, 422, and 423 represent the current selected value of the corresponding loading parameter. For example, plots 401, 411, and 421 include a vertical dashed line at a loading density of approximately 25 g/L; plots 402, 412, and 422 include a vertical dashed line at a loading buffer citrate concentration of approximately 148 mM, and plots 403, 413, and 423 show a vertical dashed line at a load % HMW of approximately 6%. The horizontal dashed lines shown in each plot 401, 402, 403, 411, 412, 413, 421, 422, and 423 represent a target performance criterion value. For example, plots 401, 402, 403, and 404 include a horizontal dashed line corresponding to a yield of approximately 80%; plots 411, 412, 413, and 414 include a horizontal dashed line corresponding to an eluate % HMW of approximately 3%; and plots 421, 422, and 423 include a horizontal dashed line corresponding to a desirability metric value of 0.9.

Using the dynamic plot, thousands of chromatography protocols were evaluated in a high-throughput manner. The dynamic plot, and in particular, the desirability plots 421, 422, and 423, can also be used to easily visualize loading parameters values that optimize desirability of a chromatography protocol. For example, based on the dynamic plot shown in FIG. 4, a chromatography protocol was developed that included the following loading parameters: a loading density of approximately 20 to approximately 30 g/L, a loading buffer citrate concentration of approximately 140 mM to approximately 155 mM, and a load % HMW of approximately 7% to approximately 15%.

### Example 3

The predictive model (e.g., including the first predictive model and the second predictive model) developed in Example 2 was verified using a full scale chromatography run. The full-scale chromatography run was performed at on a sartobind phenyl membrane at a loading density of 26.7 g/L. The citrate concentration of the loading buffer was 150 mM. The load for the full scale chromatography run included a % HMW of 9.44%.

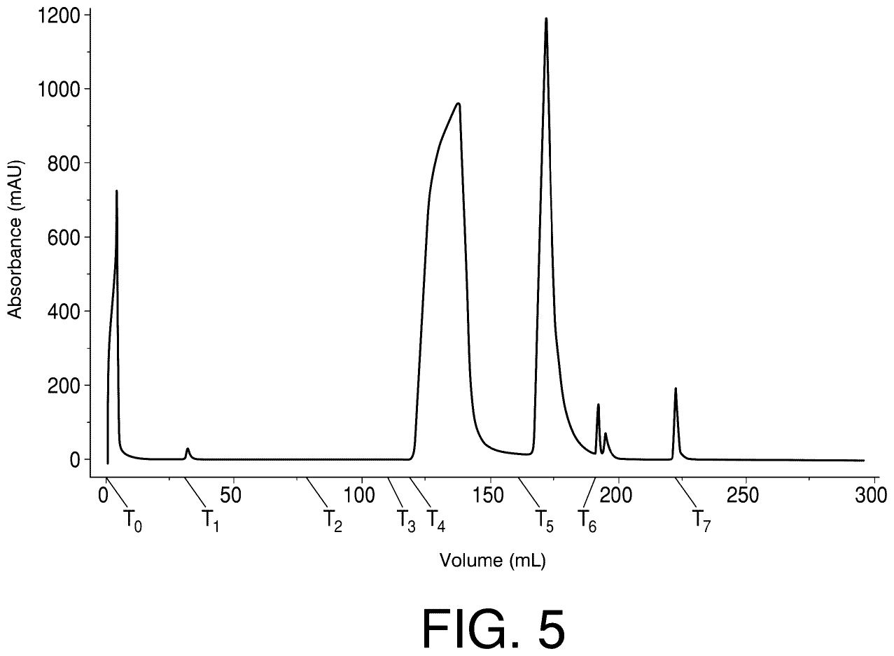

The exemplary chromatography protocol used in the full scale chromatography run is represented by the chromatogram shown in FIG. 5, where an absorbance measured at the outlet of the chromatography apparatus is plotted versus the volume passed through the apparatus. Referring to FIG. 5, at T₀, a buffer including 0.1 N sodium hydroxide was introduced into the chromatography apparatus. At, T₁, water (e.g., reverse osmosis deionized water) was introduced into the apparatus. At T₂, an equilibration buffer was introduced into the apparatus. The equilibration buffer included 50 mM Tris and 150 mM sodium citrate, and had a pH of 8.0. At T₃, the load was introduced into the chromatography apparatus. Pool collection began when the absorbance of the eluate increased after the load was introduced (e.g., at approximately T₄). At T₅, the stripping buffer was introduced into the chromatography apparatus. The stripping buffer included reverse osmosis deionized water. At T₆, a buffer including 0.5 N sodium hydroxide was introduced into the chromatography apparatus. The eluate collected between T₅ and T₆ may be referred to as the strip fraction. At T₇ a storage buffer was introduced into the chromatography apparatus. The storage buffer included 20 wt. % ethanol.

The total protein yield and eluate % HMW of the full scale chromatography run were recorded and compared to projected performance criteria values generated using the predictive model. The actual performance criteria values and projected performance criteria values are summarized in Table 4.

TABLE 4

Performance Criteria

| | Yield (%) | Eluate % HMW (%) |
|---|---|---|
| Model Prediction | 69.5 | 4.34 |
| Actual | 70.7 | 4.11 |

### Example 4

Additional studies were conducted to validate the chromatography protocol developed in Example 2. Mass spectra were generated from the load of the chromatography protocol and the eluate of the chromatography protocol from Example 3, to confirm the drug distribution of the target molecule before and after the chromatography protocol. The mass spectrum of the load is shown in FIG. 5A and the mass spectrum of the eluate is shown in FIG. 5B. The various peaks of the mass spectra correspond to species of the target molecule. For example, while each peak corresponds to the antibody-drug conjugate target molecule, each peak may correspond to a species of the target molecule with a different drug-to-antibody ratio. A summary of the peaks from the mass spectra is provided in Table 5.

TABLE 5

| | Drug-to- | Load Peak | Relative Abundance | Eluate Peak | Relative Abundance |
|---|---|---|---|---|---|
| Peak | Antibody | Location | of Species in Load | Location | of Species in Eluate |
| Number | Ratio | (FIG. 5A) | (FIG. 5A) | (FIG. 5B) | (FIG. 5B) |
| 501 | 0 | 144657.5 | 6.5% | 144657.0 | 8.6% |
| 502 | 1 | 145907.5 | 17.8% | 145908.0 | 22.7% |
| 503 | 2 | 147158.5 | 24.0% | 147158.5 | 29.0% |
| 504 | 3 | 148409.0 | 20.8% | 148409.0 | 22.9% |
| 505 | 4 | 149659.0 | 15.1% | 149660.5 | 12.7% |
| 506 | 5 | 150910.5 | 8.4% | 150912.0 | 4.1% |
| 507 | 6 | 152159.0 | 5.2% | 152151.5 | 0.0% |
| 508 | 7 | 153411.0 | 2.3% | N/A | 0.0% |

By comparing the relative abundances of the various target molecule species in a sample, the drug-to-antibody ratio of the sample may be determined. In this example, the mass spectra were used to determine the drug-to-antibody ratio of the load and the drug-to-antibody ratio of the eluate. The load had a drug-to-antibody ratio of 3.9 and the eluate had a drug-to-antibody ratio of 2.9.

The decreased drug-to-antibody ratio of the eluate, compared to the load, is evidence the higher drug-to-antibody ratio species are being retained on the membrane. Removal of higher drug-to-antibody ratio species results in a more homogeneous drug product. Further, the presence of some high drug-to-antibody ratio species may impact the stability of the resulting drug product and/or may alter the immunogenic response to the resulting drug product. Therefore, removal of high drug-to-antibody ratio species according to aspects of the present disclosure may result in a drug product with a decreased risk profile.

### Example 5

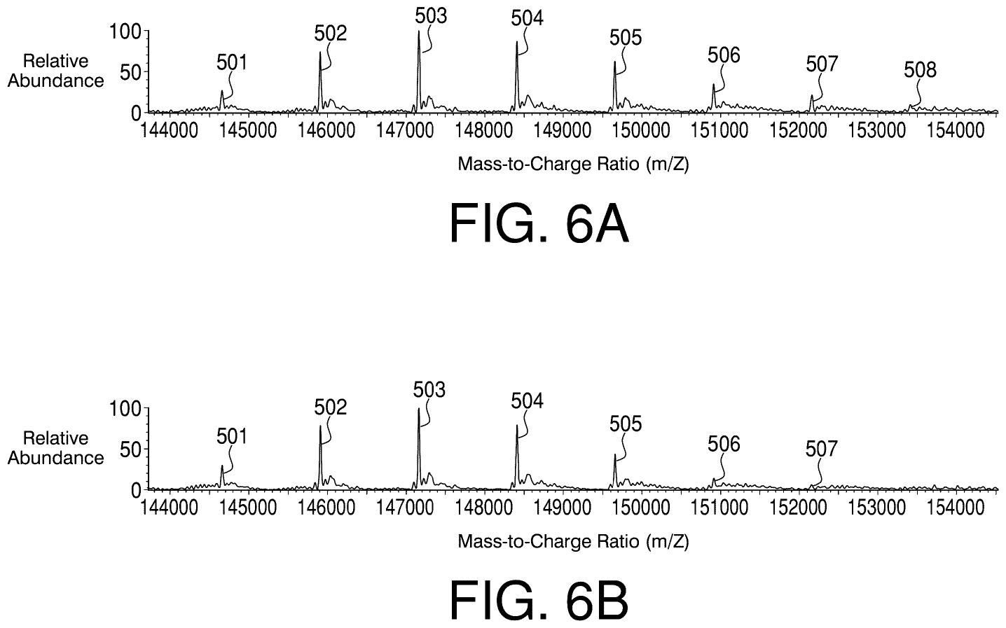

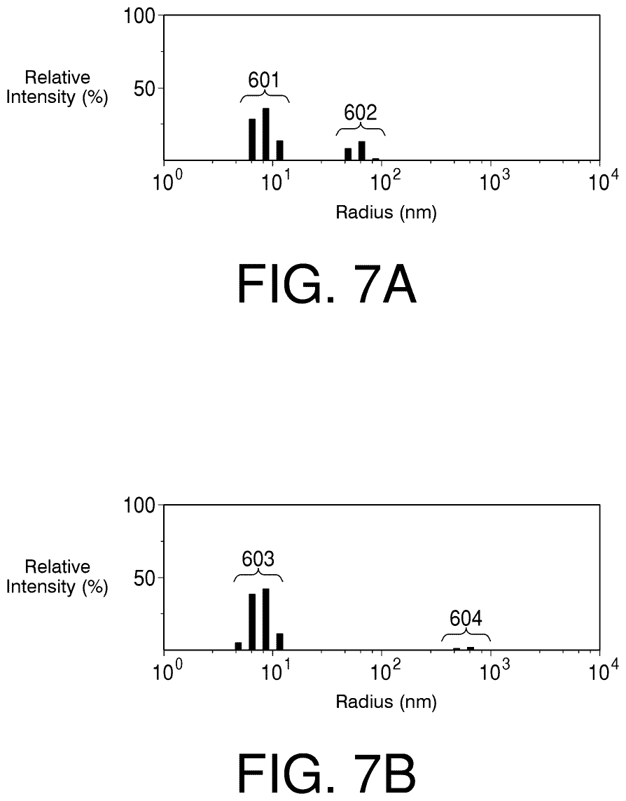

The particle size distributions of the load and eluate for the chromatography protocol from Example 3 were determined using dynamic light scattering. The measured particle size distributions for the load are shown in FIG. 6A and the particle size distributions for the eluate are shown in FIG. 6B. The peaks shown in the plots of FIGS. 6A and 6B are summarized in Table 6.

TABLE 6

| | | | Std. | Relative | Std. Deviation |
|---|---|---|---|---|---|
| FIG. | Peak | Radius | Deviation | Intensity | of Relative |
| Number | Number | (nm) | of Radius | (%) | Intensity |
| 6A | 601 | 9.0 | 0.5 | 81.5 | 2.5 |
| 6A | 602 | 63.5 | 6.0 | 17.5 | 3.2 |
| 6B | 603 | 7.6 | 0.2 | 95.2 | 1.4 |
| 6B | 604 | 211.4 | 15.8 | 3.9 | 0.8 |

As shown in FIGS. 6A, 6B, and Table 6, the developed chromatography protocol modifies particle size distribution and average particle size by removing higher sized molecules, confirming the reduction of aggregates by the chromatography protocol. This is further evidenced by the eluate % HMW of 4.11% shown in Table 4, which represents a % HMWΔ of 5.33% from the load % HMW of 9.44%.

### Example 6

As part of the validation of the chromatography protocol developed in Example 2, bioassays were performed on the load and eluate of the chromatography protocol of Example 3.

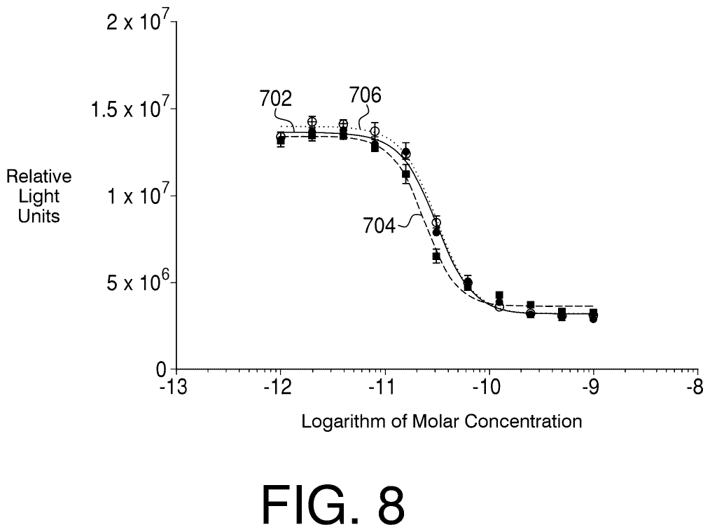

For the assay, 1300 EBC-1 human cells (endogenously expressing human MET) were plated in minimal essential medium 1× Earle's salts without non-essential amino acids, supplemented with 10% fetal bovine serum and 1% penicillin/streptomycin/glutamine in white 96-well plates with opaque bottoms. The wells were incubated overnight at 37° C. and 5% CO₂. After incubation, 20 μL serial dilutions of the load from the chromatography protocol of Example 3, the eluate of the chromatography protocol, and the conjugated references standard were prepared in the media as the incubation step with concentrations ranging from 1.0 pM to 1.0 nM. The serial dilutions were added to the 96-well plate and incubated (i.e., a secondary incubation) for 72 hours. After the secondary incubation, 100 μL of a commercially available cell viability detection reagent was added to the plates, and the luminescence was recorded using an ENVISION Multimode Plate Reader. The plot of the bioassay results is shown in FIG. 8, where line 702 represents measurements from the reference standard, line 704 represents measurements from the load of the chromatography protocol described in Example 3, and line 706 represents measurements from the eluate of the chromatography protocol. The plot in FIG. 8 shows that the cytotoxic properties of the antibody-drug conjugate from the eluate of the chromatography protocol is consistent with those properties of lots generated using previously validated methods.

The data from the bioassay were analyzed using a 4-parameter logistic equation over an 11-point response curve with GraphPad Prism software. Half-maximal inhibitory concentration (IC50) values, were calculated and used to determine relative potency values using Equation 6, shown below.

$$Relative Potency = \frac{{IC}_{50} Reference Standard}{{IC}_{50} Test Article} \times 100 \qquad Equation 6$$

Referring to Equation 6, IC₅₀ Reference Standard refers to the IC₅₀ of the reference standard and IC₅₀ Test Article refers to the IC₅₀ of the sample being tested (e.g., a load or an eluate of a chromatography operation). The relative potencies from the bioassay of Example 6 are summarized in Table 7.

### Example 7

As part of the validation of the chromatography protocol developed in Example 2, enzyme-linked immunosorbent assay (ELISA) binding assays were performed on the load and eluate of the chromatography protocol of Example 3.

An ELISA plate was coated with unconjugated anti-human MET antibody was incubated overnight at 4° C. Following overnight incubation, serial dilutions of antibody-drug conjugates described in Example 1, and serial dilutions of a reference standard, were pre-bound to 50 pM of human MET myc-myc-hexahistidine at room temperature for one hour. The reference standard was a lot of antibody-drug conjugates that were manufactured using a previously validated methods.

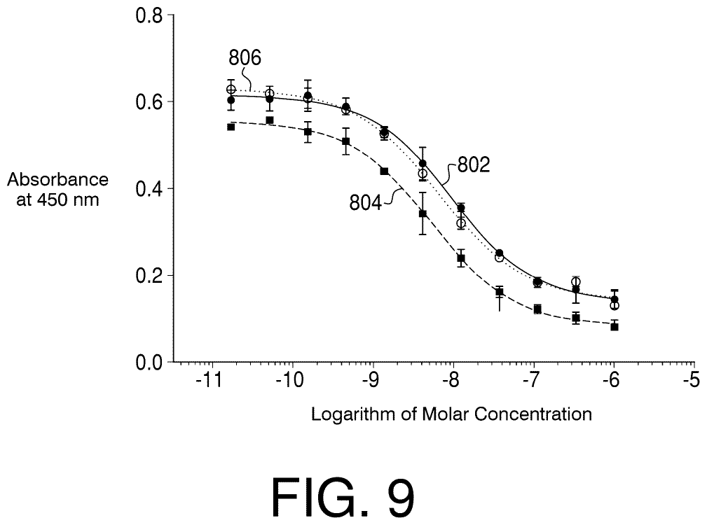

Each pre-bound mix was then transferred in duplicate to the ELISA plate coated with unconjugated anti-human MET antibody (i.e., the same antibody used in the antibody-drug conjugate), and incubated for one hour. After incubation, the plate-bound human MET myc-myc-hexahistidine was detected by horseradish peroxidase (HRP) conjugated to an anti-histidine antibody (such as those commercially available from Qiagen™ (cat. no. 34460)), and visualized using 3,3′,5,5′-tetramethylbenzidine (TMB), a colorimetric HRP substrate. The absorbance for each well of the ELISA plate at 450 nm was recorded on an ENVISION Multimode Plate Reader and plotted as a function of antibody concentrations. The plot of the bioassay results is shown in FIG. 9, where line 802 represents measurements from the reference standard, line 804 represents measurements from the load of the chromatography protocol described in Example 3, and line 806 represents measurements from the eluate of the chromatography protocol described in Example 3. The plot in FIG. 9 shows that the relative binding potencies of the conjugates from the load and eluate of the chromatography protocol are not markedly different than the binding potency of the reference standard (e.g., a lot manufactured using a previously validated process).

The data from the bioassay were analyzed using a 4-parameter logistic equation over an 11-point response curve with GraphPad Prism software. Half-maximal inhibitory concentration (IC50) values, defined as the concentration of the antibody-drug conjugate required to block 50% of human MET myc-myc-hexahistidine binding to the plate coated with unconjugated antibody, were derived from the analysis and used to calculate the relative potency values using Equation 6.

The relative potencies from the bioassay of Example 6 and the binding assay of Example 7 are summarized in Table 7.

TABLE 7

Relative Potency

| | Bioassay | Binding Assay |
|---|---|---|
| Test Article | (Example 6) | (Example 7) |
| Load | 134% | 167% |
| Eluate | 99% | 156% |

The present disclosure is further described by the following non-limiting items.

Item 1. A method of generating a chromatography protocol for a target molecule, the method comprising:
identifying chromatography loading parameters;
identifying chromatography performance criteria;
generating a domain of potential predictive models relating the chromatography loading parameters to the chromatography performance criteria;
selecting combinations of test values of the loading parameters, wherein the selected combinations of test values form a set of test value combinations;
conducting a chromatography run for each combination of the set of test values combinations, thereby generating actual performance criteria values corresponding to each combination of the set of test value combinations; and
ranking each predictive model, of the domain of predictive models, based on a correlation of performance criteria values predicted by the model to the actual performance criteria values;
wherein the target molecule is an antibody-drug conjugate.

Item 2. The method of item 1, wherein the chromatography loading parameters include a loading buffer salt concentration, a chromatography media density, a high molecular weight species content of the load, or a combination thereof.

Item 3. The method of any one of items 1 or 2, wherein the chromatography performance criteria include a high molecular weight species content reduction, yield of a target molecule, or a combination thereof.

Item 4. The method of any one of items 1 to 3, further comprising: selecting a predictive model that has the highest rank; and using the selected model to determine values of the chromatography loading parameters for the chromatography protocol.

Item 5. The method of item 4, wherein the selected model predicts that the values of the chromatography loading parameters for the chromatography protocol correspond to one or more target performance criteria values.

Item 6. The method of any one of items 1 to 5, further comprising, developing a desirability metric that includes a quantitative relationship between performance criteria.

Item 7. The method of item 6, wherein the desirability metric is calculated as a composite of two or more performance criteria, and wherein each performance criterion is assigned a weight that contributes to the desirability metric.

Item 8. The method of item 7, wherein the two or more performance criteria include a high molecular weight species content reduction and a yield of a target molecule, and wherein each performance criterion is equally weighted.

Item 9. A method of generating a chromatography protocol for a target molecule, the method comprising:
identifying a first chromatography parameter and a second chromatography parameter;
identifying a first performance criterion and a second performance criterion;
selecting first test values for the first chromatography parameter;
selecting second test values for the second chromatography parameter;
identifying a chromatography media;
generating first performance criterion values, wherein each first performance criterion value corresponds to a combination of a first test value and a second test value;
generating second performance criterion values, wherein each second performance criterion value corresponds to a combination of a first test value and a second test value;
generating a first pool of multivariate models, wherein each multivariate model of the first pool of multivariate models relates the first and second chromatography parameters to the first performance criterion;
generating a second pool of multivariate models, wherein each multivariate model of the second pool of multivariate models relates the first and second chromatography parameters to the second performance criterion;
generating first projected performance criterion values using each multivariate model of the first pool of multivariate models, wherein each first projected performance criterion value corresponds to a combination of a first test value and a second test value;
generating second projected performance criterion values using each multivariate model of the second pool of multivariate models, wherein each second projected performance criterion value corresponds to a combination of a first test value and a second test value;
determining a coefficient of determination for a multivariate model in the first pool of multivariate models; and
determining a coefficient of determination for a multivariate model in the second pool of multivariate models;
wherein the target molecule is an antibody-drug conjugate.

Item 10. The method of item 9, wherein identifying the chromatography media comprises:
performing a first chromatography run on a first chromatography media to generate a first desirability value corresponding to the first chromatography media;
performing a second chromatography run on a second chromatography media to generate a second desirability value corresponding to the second chromatography media; and selecting the chromatography media with the that corresponds to the greatest desirability value.

Item 11. The method of item 10, wherein the first chromatography run and the second chromatography run are conducted with the same media density and loading buffer composition.

Item 12. The method of item 11, wherein the first desirability value is calculated based on one or more performance criteria values of the first chromatography run, and the second desirability value is calculated based on one or more performance criteria values of the second chromatography run.

Item 13. The method of any one of items 9 to 12, wherein the first chromatography parameter includes a column loading of a HIC media (g/L), the second chromatography parameter includes a citrate salt concentration of a loading buffer, the first performance criterion is a yield, and the second performance criterion is a quantification of the reduction of an impurity.

Item 14. The method of any one of items 9 to 13, wherein generating the first pool of multivariate models includes:
identifying a domain of potential multivariate models;
calculating a variance inflation factor for each potential multivariate model of the domain of potential multivariate models;
selecting all potential multivariate models with a variance inflation factor less than or equal to a collinearity threshold to generate the first pool of multivariate models.

Item 15. A method of purifying a target molecule, the method comprising:
introducing a load including a high molecular weight species concentration (% HMW) of approximately 3 percent to approximately 20 percent to a chromatography apparatus comprising sartobind phenyl chromatography media, wherein the load is introduced at a density of approximately 10 grams of load per liter of total volume of the chromatography apparatus to approximately 40 grams per liter, and wherein the load comprises the target molecule and approximately 5 mM to approximately 200 mM of citrate; and
passing an eluate comprising the target molecule from the chromatography apparatus, wherein a yield of the target molecule in the eluate is at least approximately 70%, and wherein a difference of the % HMW of load and a % HMW of the eluate is at least approximately 2%;
wherein the target molecule is an antibody-drug conjugate.

Item 16. The method of item 15, the load is introduced at a density of approximately 20 grams per liter to approximately 30 grams per liter.

Item 17. The method of any one of item 15, wherein the antibody-drug conjugate includes a drug conjugated to an antibody via lysine conjugation.

Item 18. The method of any one of items 15-17, wherein:
the % HMW of the load is approximately 7% to approximately 15%;
the load comprises approximately 110 mM to approximately 175 mM citrate;
the yield of the target molecule in the eluate is approximately 75% to approximately 95%; and/or
the difference of the % HMW of load and a % HMW of the eluate is approximately 3% to approximately 8%.

Item 19. The method of item 15, wherein the antibody-drug conjugate includes a cleavable maytansinoid.

Item 20. The method of item 15, wherein the antibody-drug conjugate includes an IgG 4 antibody.

Those skilled in the art will appreciate that the conception upon which this disclosure is based may readily be used as a basis for designing other methods and systems for carrying out the several purposes of the present disclosure. Accordingly, the claims are not to be considered as limited by the foregoing description.

What is claimed is:

1. A method of generating a chromatography protocol for a target molecule, the method comprising:
identifying chromatography loading parameters;
identifying chromatography performance criteria;
generating a domain of potential predictive models relating the chromatography loading parameters to the chromatography performance criteria;
selecting combinations of test values of the loading parameters, wherein the selected combinations of test values form a set of test value combinations;
conducting a chromatography run for each combination of the set of test values combinations, thereby generating actual performance criteria values corresponding to each combination of the set of test value combinations; and
ranking each predictive model, of the domain of predictive models, based on a correlation of performance criteria values predicted by the model to the actual performance criteria values;
wherein the target molecule is an antibody-drug conjugate.

2. The method of claim 1, wherein the chromatography loading parameters include a loading buffer salt concentration, a chromatography media density, a high molecular weight species content of the load, or a combination thereof.

3. The method of claim 1, wherein the chromatography performance criteria include a high molecular weight species content reduction, yield of a target molecule, or a combination thereof.

4. The method of claim 1, further comprising:
selecting a predictive model that has the highest rank; and
using the selected model to determine values of the chromatography loading parameters for the chromatography protocol.

5. The method of claim 4, wherein the selected model predicts that the values of the chromatography loading parameters for the chromatography protocol correspond to one or more target performance criteria values.

6. The method of claim 1, further comprising, developing a desirability metric that includes a quantitative relationship between performance criteria.

7. The method of claim 6, wherein the desirability metric is calculated as a composite of two or more performance criteria, and wherein each performance criterion is assigned a weight that contributes to the desirability metric.

8. The method of claim 7, wherein the two or more performance criteria include a high molecular weight species content reduction and a yield of a target molecule, and wherein each performance criterion is equally weighted.

9. A method of generating a chromatography protocol for a target molecule, the method comprising:
identifying a first chromatography parameter and a second chromatography parameter;
identifying a first performance criterion and a second performance criterion;
selecting first test values for the first chromatography parameter;
selecting second test values for the second chromatography parameter;
identifying a chromatography media;
generating first performance criterion values, wherein each first performance criterion value corresponds to a combination of a first test value and a second test value;
generating second performance criterion values, wherein each second performance criterion value corresponds to a combination of a first test value and a second test value;
generating a first pool of multivariate models, wherein each multivariate model of the first pool of multivariate models relates the first and second chromatography parameters to the first performance criterion;
generating a second pool of multivariate models, wherein each multivariate model of the second pool of multivariate models relates the first and second chromatography parameters to the second performance criterion;
generating first projected performance criterion values using each multivariate model of the first pool of multivariate models, wherein each first projected performance criterion value corresponds to a combination of a first test value and a second test value;
generating second projected performance criterion values using each multivariate model of the second pool of multivariate models, wherein each second projected performance criterion value corresponds to a combination of a first test value and a second test value;
determining a coefficient of determination for a multivariate model in the first pool of multivariate models; and
determining a coefficient of determination for a multivariate model in the second pool of multivariate models;
wherein the target molecule is an antibody-drug conjugate.

10. The method of claim 9, wherein identifying the chromatography media comprises:
performing a first chromatography run on a first chromatography media to generate a first desirability value corresponding to the first chromatography media;
performing a second chromatography run on a second chromatography media to generate a second desirability value corresponding to the second chromatography media; and
selecting the chromatography media with the that corresponds to the greatest desirability value.

11. The method of claim 10, wherein the first chromatography run and the second chromatography run are conducted with the same media density and loading buffer composition.

12. The method of claim 11, wherein the first desirability value is calculated based on one or more performance criteria values of the first chromatography run, and the second desirability value is calculated based on one or more performance criteria values of the second chromatography run.

13. The method of claim 9, wherein the first chromatography parameter includes a column loading of a HIC media (g/L), the second chromatography parameter includes a citrate salt concentration of a loading buffer, the first performance criterion is a yield, and the second performance criterion is a quantification of the reduction of an impurity.

14. The method of claim 9, wherein generating the first pool of multivariate models includes:
identifying a domain of potential multivariate models;
calculating a variance inflation factor for each potential multivariate model of the domain of potential multivariate models;
selecting all potential multivariate models with a variance inflation factor less than or equal to a collinearity threshold to generate the first pool of multivariate models.

15. A method of purifying a target molecule, the method comprising:
introducing a load including a high molecular weight species concentration (% HMW) of approximately 3 percent to approximately 20 percent to a chromatography apparatus comprising sartobind phenyl chromatography media, wherein the load is introduced at a density of approximately 10 grams of load per liter of total volume of the chromatography apparatus to approximately 40 grams per liter, and wherein the load comprises the target molecule and approximately 5 mM to approximately 200 mM of citrate; and
passing an eluate comprising the target molecule from the chromatography apparatus, wherein a yield of the target molecule in the eluate is at least approximately 70%, and wherein a difference of the % HMW of load and a % HMW of the eluate is at least approximately 2%;
wherein the target molecule is an antibody-drug conjugate.

16. The method of claim 15, wherein the load is introduced at a density of approximately 20 grams per liter to approximately 30 grams per liter.

17. The method of claim 15, wherein the antibody-drug conjugate includes a drug conjugated to an antibody via lysine conjugation.

18. The method of claim 15, wherein:
the % HMW of the load is approximately 7% to approximately 15%;
the load comprises approximately 110 mM to approximately 175 mM citrate;
the yield of the target molecule in the eluate is approximately 75% to approximately 95%; and/or
the difference of the % HMW of load and a % HMW of the eluate is approximately 3% to approximately 8%.

19. The method of claim 15, wherein the antibody-drug conjugate includes a cleavable maytansinoid.

20. The method of claim 15, wherein the antibody-drug conjugate includes an IgG 4 antibody.
